(19) United States
(12) Patent Application Publication
(10) Pub. No.: US 2024/0343692 A1
(43) Pub. Date: Oct. 17, 2024

(54) SELECTIVE PSYCHEDELIC COMPOUNDS

(71) Applicant: University of Pittsburgh - Of the Commonwealth System of Higher Education, Pittsburgh, PA (US)

(72) Inventors: Peter Wipf, Pittsburgh, PA (US); Nikhil R. Tasker, Pittsburgh, PA (US); Ethan J. Pazur, Dearborn, MI (US)

(73) Assignee: University of Pittsburgh - Of the Commonwealth System of Higher Education, Pittsburgh, PA (US)

(21) Appl. No.: 18/682,031

(22) PCT Filed: Jun. 21, 2022
(86) PCT No.: PCT/US2022/034290

§ 371 Date: Feb. 7, 2024

Related U.S. Application Data
(60) Provisional application No. 63/231,053, filed on Aug. 9, 2021.

(51) Int. Cl. C07D 221/22; A61K 31/4045; A61K 31/437; A61K 31/439; C07D 221/18

(52) U.S. Cl. CPC C07D 221/22; A61K 31/4045; A61K 31/437; A61K 31/439; C07D 221/18

(57) ABSTRACT

A compound, or a pharmaceutically acceptable salt thereof, of formula I:

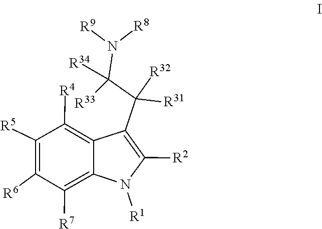

wherein R¹, R⁸, R⁹, R³¹, R³², R³³, and R³⁴ are each independently H, C₁-C₆ alkyl, or substituted C₁-C₆ alkyl; and
R², R⁴, R⁵, R⁶, and R⁷ are each independently H, F, Cl, Br, —OR¹—NR⁸R⁹, C₁-C₆ alkyl, or substituted C₁-C₆ alkyl.

This application claims the benefit of U.S. Provisional Appl. 63/231,053, filed Aug. 9, 2021, which is incorporated herein by reference.

### BACKGROUND

Synthetic and pharmacological investigations of ergot alkaloids have persisted for nearly a century due to their profound biological effects and potential to treat mood disorders and cognitive ailments. Recent clinical research has focused primarily on _{D}-lysergic acid diethylamide (_{D}-LSD) as a potential medication for depression, anxiety, neurodegeneration, and substance use disorders. However, given the strong hallucinogenic properties and potential side effects associated with _{D}-LSD, establishing effective dosing regimens has remained an ongoing challenge. In contrast to the investigations of lysergic acid amides, the therapeutic potential of many other ergot alkaloids has remained obscure, in spite of their frequently more selective serotonin receptor agonism. For example, (+)- and (−)-cycloclavine were found to show increased selectivity for dopamine D3 and serotonin 5-HT1A and 5-HT2A CNS receptors, and indole fluorination of (+)-lysergol further enhanced selectivity among 5-HT receptors compared to _{D}-LSD. Selective agonism at 5-HT₁₋₇ receptors is a target profile for the treatment of neuropsychiatric conditions, with the potential to yield new classes of therapeutics.

### SUMMARY

Disclosed herein are compounds, or pharmaceutically acceptable salts thereof, of formula I:

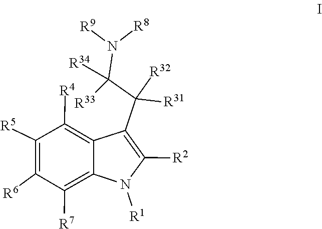

wherein R¹, R⁸, R⁹, R³¹, R³², R³³, and R³⁴ are each independently H, C₁-C₆ alkyl, or substituted C₁-C₆ alkyl; and
R², R⁴, R⁵, R⁶, and R⁷ are each independently H, F, Cl, Br, —OR¹, —NR⁸R⁹, C₁-C₆ alkyl, or substituted C₁-C₆ alkyl.

Disclosed herein are compounds, or pharmaceutically acceptable salts thereof, of formula II:

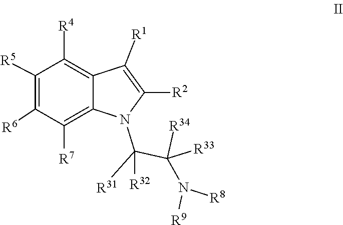

wherein R¹, R⁸, R⁹, R³¹, R³², R³³, and R³⁴ are each independently H, C₁-C₆ alkyl, or substituted C₁-C₆ alkyl; and
R², R⁴, R⁵, R⁶, and R⁷ are each independently H, F, Cl, Br, —OR¹, —NR⁸R⁹, C₁-C₆ alkyl, or substituted C₁-C₆ alkyl.

Disclosed herein are compounds, or pharmaceutically acceptable salts thereof, of formula III:

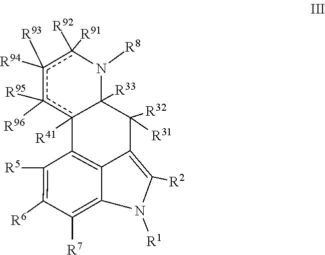

wherein R¹ and R⁸ are each independently H, C₁-C₆ alkyl, or substituted C₁-C₆ alkyl;
R², R⁵, R⁶, R⁷, R⁴¹, R⁹³, R⁹⁴, R⁹⁵, and R⁹⁶ are each independently H, F, Cl, Br, —OR¹, —NR⁸R⁹, —CR¹R¹OR¹, —CO₂R¹, —CONR⁸R⁹, C₁-C₆ alkyl, or substituted C₁-C₆ alkyl;
R⁹, R³¹, R³², R³³, R⁹¹, and R⁹² are each independently H, —CR¹R¹OR¹, —CO₂R¹, —CONR⁸R⁹, C₁-C₆ alkyl, or substituted C₁-C₆ alkyl; and
where each bond represented by [symbol] is a single or double bond as needed to satisfy valence requirements, and where each of R⁴¹, R⁹⁵, R⁹⁴, and R⁹¹ may or may not be present as needed to satisfy valency requirements.

Disclosed herein are compounds, or pharmaceutically acceptable salts thereof, of formula IV:

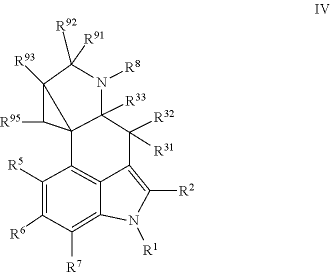

wherein R¹ and R⁸ are each independently H, C₁-C₆ alkyl, or substituted C₁-C₆ alkyl;
R², R⁵, R⁶, R⁷, R⁹³, and R⁹⁵ are each independently H, F, Cl, Br, —OR¹, —NR⁸R⁹, —CR¹R¹OR¹, —CO₂R¹, —CONR⁸R⁹, C₁-C₆ alkyl, or substituted C₁-C₆ alkyl; and
R⁹, R³¹, R³², R³³, R⁹¹, and R⁹² are each independently H, —CR¹R¹OR¹, —CO₂R¹, —CONR⁸R⁹ , C₁-C₆ alkyl, or substituted C₁-C₆ alkyl.

Disclosed herein are compounds, or pharmaceutically acceptable salts thereof, of formula V:

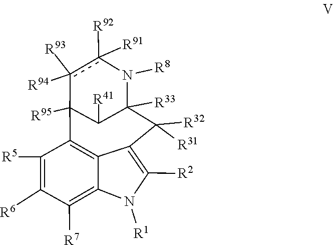

wherein R¹ and R⁸ are each independently H, C₁-C₆ alkyl, or substituted C₁-C₆ alkyl;
R², R³, R⁶, R⁷, R⁴¹, R⁹³, R⁹⁴ and R⁹⁵ are each independently H, F, Cl, Br, —OR¹, —NR⁸R⁹, —CR¹R¹OR¹, —CO₂R¹, —CONR⁸R⁹, C₁-C₆ alkyl, or substituted C₁-C₆ alkyl;
R⁹, R³¹, R³², R³³, R⁹¹, and R⁹² are each independently H, —CR¹R¹OR¹, —CO₂R¹, —CONR⁸R⁹, C₁-C₆ alkyl, or substituted C₁-C₆ alkyl; and
where each bond represented by [symbol] is a single or double bond as needed to satisfy valence requirements, and where each of R⁹⁴ and R⁹¹ may or may not be present as needed to satisfy valency requirements.

The foregoing will become more apparent from the following detailed description, which proceeds with reference to the accompanying figures.

### BRIEF DESCRIPTION OF THE DRAWINGS

FIG. 1. Histogram for 5-HT1A(h) (agonist radioligand).

FIG. 2. Histogram for 5-HT2A(h) (agonist radioligand).

FIG. 3. Histogram for 5-HT2B(h) (agonist radioligand).

FIG. 4. Histogram for 5-HT2C(h) (agonist radioligand).

FIGS. 5-10. HPLC Chromatograms. Lysergols were analyzed on a Daicel Chiralcel OD-H column (46 mm D×250 mm L, ×5 μm) using 7:3 Hex:IPA at 0.5 mL/min. These conditions were translated to a semi-prep Daicel Chiralcel OD-H column (20 mm D×250 mm L, ×5 μm) using 7:3 Hex:IPA at 6.0 mL/min to separate the enantiomers.

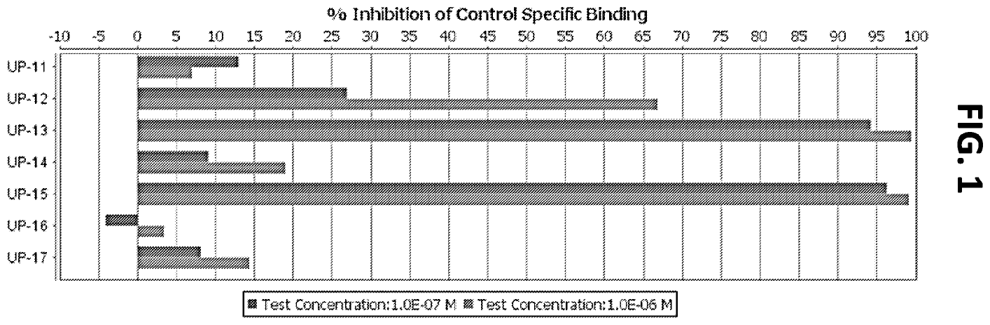

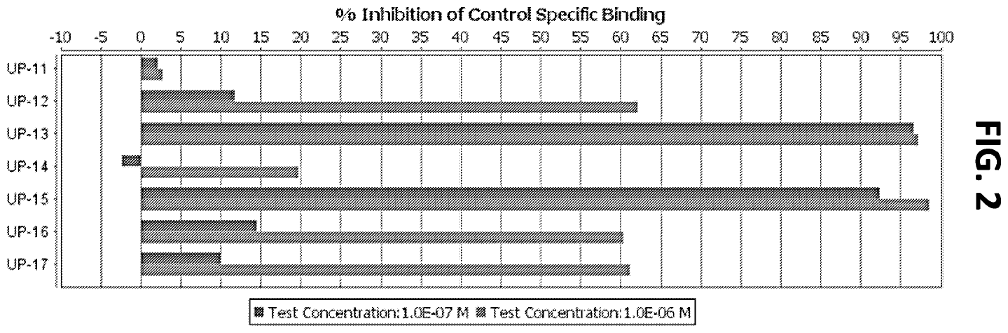

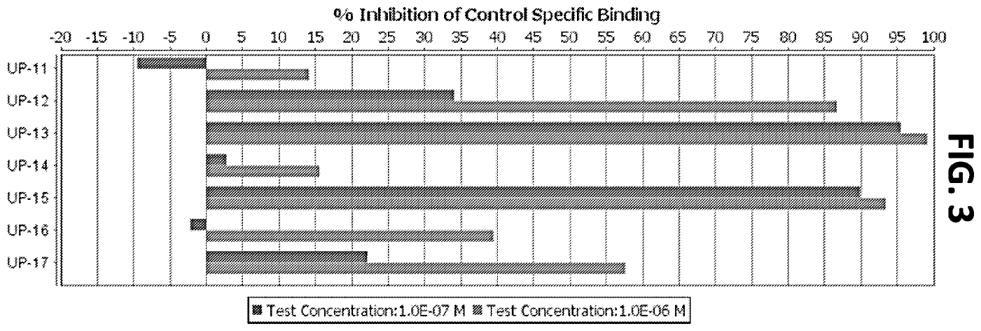

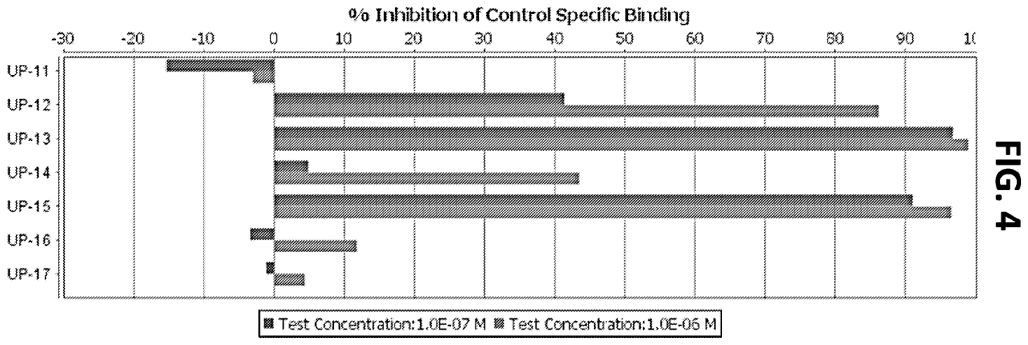

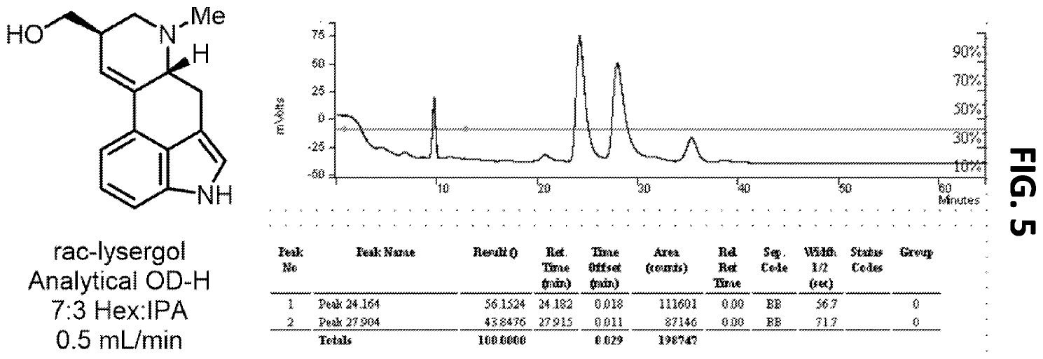

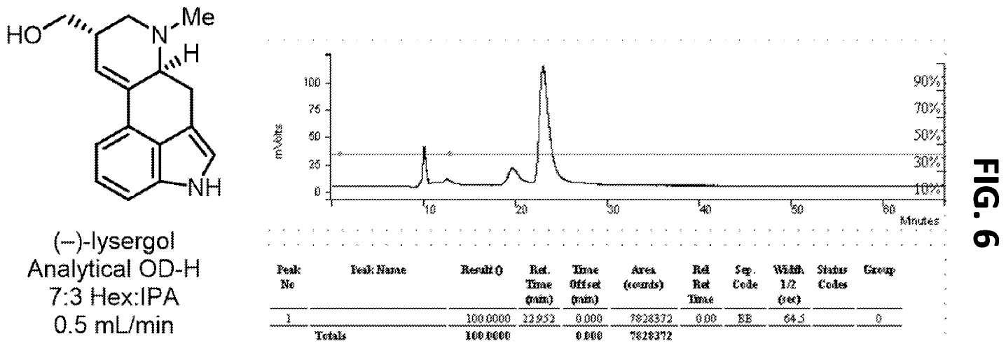

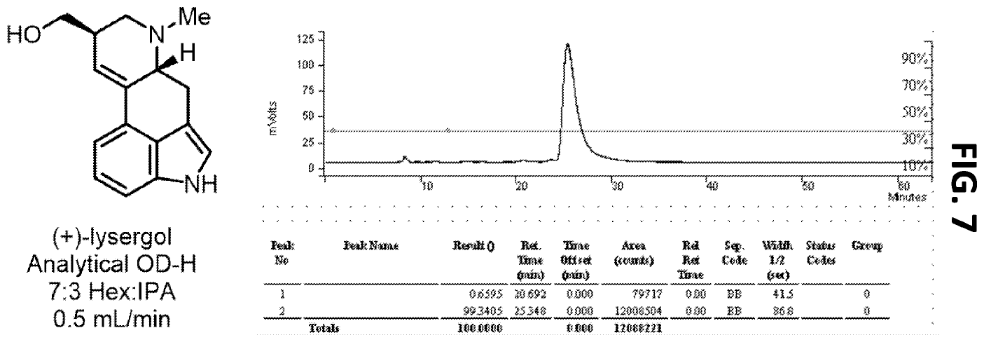

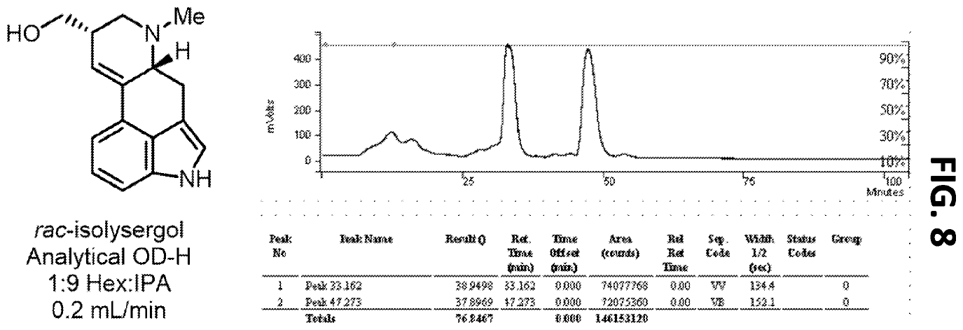

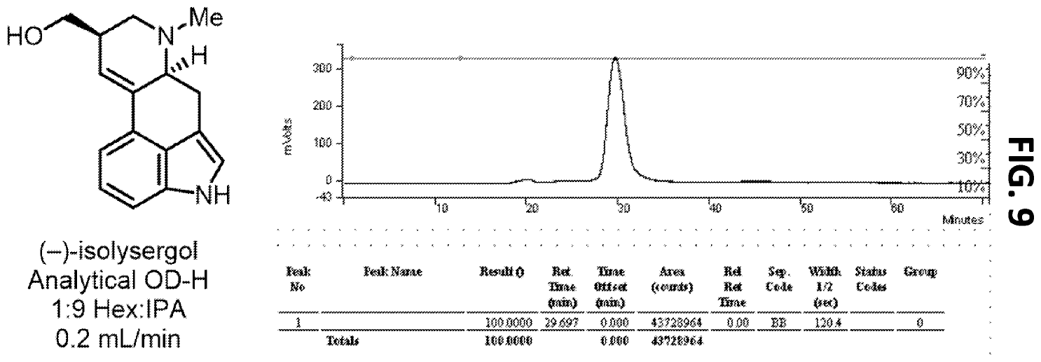

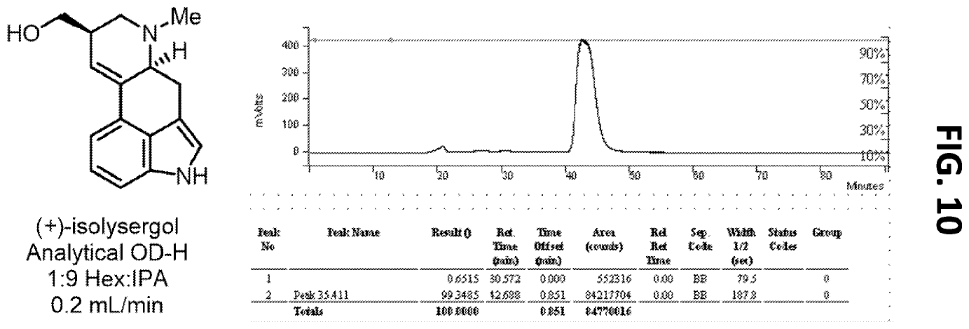

### DETAILED DESCRIPTION

### Terminology

The following explanations of terms and methods are provided to better describe the present compounds, compositions and methods, and to guide those of ordinary skill in the art in the practice of the present disclosure. It is also to be understood that the terminology used in the disclosure is for the purpose of describing particular embodiments and examples only and is not intended to be limiting.

“Administration” as used herein is inclusive of administration by another person to the subject or self-administration by the subject.

The term “alkyl” refers to a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is a saturated branched or unbranched hydrocarbon having from 1 to 6 carbon atoms. Preferred alkyl groups have 1 to 4 carbon atoms. Alkyl groups may be “substituted alkyls” wherein one or more hydrogen atoms are substituted with a substituent such as halogen, cycloalkyl, alkoxy, amino, hydroxyl, aryl, alkenyl, or carboxyl. For example, a lower alkyl or (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloalkyl (C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₆)alkanoyl can be acetyl, propanoyl or butanoyl; halo(C₁-C₆)alkyl can be iodomethyl, bromomethyl, chloromethyl, fluoromethyl, trifluoromethyl, 2-chloroethyl, 2-fluoroethyl, 2,2,2-trifluoroethyl, or pentafluoroethyl; hydroxy (C₁-C₆)alkyl can be hydroxymethyl, 1-hydroxyethyl, 2-hydroxyethyl, 1-hydroxypropyl, 2-hydroxypropyl, 3-hydroxypropyl, 1-hydroxybutyl, 4-hydroxybutyl, 1-hydroxypentyl, 5-hydroxypentyl, 1-hydroxyhexyl, or 6-hydroxyhexyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₁-C₆)alkylthio can be methylthio, ethylthio, propylthio, isopropylthio, butylthio, isobutylthio, pentylthio, or hexylthio; (C₂-C₆)alkanoyloxy can be acetoxy, propanoyloxy, butanoyloxy, isobutanoyloxy, pentanoyloxy, or hexanoyloxy.

An “animal” refers to living multi-cellular vertebrate organisms, a category that includes, for example, mammals and birds. The term mammal includes both human and non-human mammals. Similarly, the term “subject” includes both human and non-human subjects, including birds and non-human mammals. Illustrative non-human mammals include animal models (such as mice), non-human primates, companion animals (such as dogs and cats), livestock (such as pigs, sheep, cows), as well as non-domesticated animals, such as the big cats. The term subject applies regardless of the stage in the organism's life-cycle. Thus, the term subject applies to an organism in utero or in ovo, depending on the organism (that is, whether the organism is a mammal or a bird, such as a domesticated or wild fowl).

“Aryl” refers to a monovalent unsaturated aromatic carbocyclic group having a single ring (e.g., phenyl) or multiple condensed rings (e.g., naphthyl or anthryl), which can optionally be unsubstituted or substituted. A “heteroaryl group,” is defined as an aromatic group that has at least one heteroatom incorporated within the ring of the aromatic group. Examples of heteroatoms include, but are not limited to, nitrogen, oxygen, sulfur, and phosphorous. Heteroaryl includes, but is not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl, and the like. The aryl or heteroaryl group can be substituted with one or more groups including, but not limited to, alkyl, alkynyl, alkenyl, aryl, halide, nitro, amino, ester, ketone, aldehyde, hydroxy, carboxylic acid, or alkoxy, or the aryl or heteroaryl group can be unsubstituted.

The term “co-administration” or “co-administering” refers to administration of a compound disclosed herein with at least one other therapeutic agent or therapy within the same general time period, and does not require administration at the same exact moment in time (although co-administration is inclusive of administering at the same exact moment in time). Thus, co-administration may be on the same day or on different days, or in the same week or in different weeks. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent and/or lowers the frequency of administering the potentially harmful (e.g., toxic) agent. “Co-administration” or “co-administering” encompass administration of two or more active agents to a subject so that both the active agents and/or their metabolites are present in the subject at the same time. Co-administration includes simultaneous administration in separate compositions, administration at different times in separate compositions, or administration in a composition in which two or more active agents are present.

“Inhibiting” refers to inhibiting the full development of a disease or condition. “Inhibiting” also refers to any quantitative or qualitative reduction in biological activity or binding, relative to a control.

“N-heterocyclic” refers to mono or bicyclic rings or ring systems that include at least one nitrogen heteroatom. The rings or ring systems generally include 1 to 9 carbon atoms in addition to the heteroatom(s) and may be saturated, unsaturated or aromatic (including pseudoaromatic). The term “pseudoaromatic” refers to a ring system which is not strictly aromatic, but which is stabilized by means of delocalization of electrons and behaves in a similar manner to aromatic rings. Aromatic includes pseudoaromatic ring systems, such as pyrrolyl rings.

Examples of 5-membered monocyclic N-heterocycles include pyrrolyl, H-pyrrolyl, pyrrolinyl, pyrrolidinyl, oxazolyl, oxadiazolyl, (including 1,2,3 and 1,2,4 oxadiazolyls) isoxazolyl, furazanyl, thiazolyl, isothiazolyl, pyrazolyl, pyrazolinyl, pyrazolidinyl, imidazolyl, imidazolinyl, triazolyl (including 1,2,3 and 1,3,4 triazolyls), tetrazolyl, thiadiazolyl (including 1,2,3 and 1,3,4 thiadiazolyls), and dithiazolyl. Examples of 6-membered monocyclic N-heterocycles include pyridyl, pyrimidinyl, pyridazinyl, pyrazinyl, piperidinyl, morpholinyl, thiomorpholinyl, piperazinyl, and triazinyl. The heterocycles may be optionally substituted with a broad range of substituents, and preferably with C₁₋₆ alkyl, C₁₋₆ alkoxy, C₂₋₆ alkenyl, C₂₋₆ alkynyl, halo, hydroxy, mercapto, trifluoromethyl, amino, cyano or mono or di(C₁₋₆alkyl)amino. The N-heterocyclic group may be fused to a carbocyclic ring such as phenyl, naphthyl, indenyl, azulenyl, fluorenyl, and anthracenyl.

Examples of 8, 9 and 10-membered bicyclic heterocycles include 1H thieno[2,3-c]pyrazolyl, indolyl, isoindolyl, benzoxazolyl, benzothiazolyl, benzisoxazolyl, benzisothiazolyl, benzimidazolyl, indazolyl, isoquinolinyl, quinolinyl, quinoxalinyl, purinyl, cinnolinyl, phthalazinyl, quinazolinyl, quinoxalinyl, benzotriazinyl, and the like. These heterocycles may be optionally substituted, for example with C₁₋₆ alkyl, C₁₋₆ alkoxy, C₂₋₆ alkenyl, C₂₋₆ alkynyl, halo, hydroxy, mercapto, trifluoromethyl, amino, cyano or mono or di(C₁₋₆alkyl)amino. Unless otherwise defined optionally substituted N-heterocyclics includes pyridinium salts and the N-oxide form of suitable ring nitrogens.

As used herein, a “psychedelic agent” refers to a compound capable of inducing an altered state of consciousness, i.e., a marked deviation in the subjective experience or psychological functioning of a normal individual from his or her usual waking consciousness. Altered states of consciousness can be monitored, evaluated, and/or quantified using any of a variety of methods known in the art including, without limitation, Dittrich's APZ (Abnormal Mental States) questionnaire, and its revised versions, OAV and 5D-ASC (see, for example, Dittrich et al., A Pharmacopsychiatry 1998, 31:80; Studerus et al., PLOS ONE 2010, 5).

The term “subject” includes both human and non-human subjects, including birds and non-human mammals, such as non-human primates, companion animals (such as dogs and cats), livestock (such as pigs, sheep, cows), as well as non-domesticated animals, such as the big cats. The term subject applies regardless of the stage in the organism's life-cycle. Thus, the term subject applies to an organism in utero or in ovo, depending on the organism (that is, whether the organism is a mammal or a bird, such as a domesticated or wild fowl).

“Substituted” or “substitution” refers to replacement of a hydrogen atom of a molecule or an R-group with one or more additional R-groups. Unless otherwise defined, the term “optionally-substituted” or “optional substituent” as used herein refers to a group which may or may not be further substituted with 1, 2, 3, 4 or more groups, preferably 1, 2 or 3, more preferably 1 or 2 groups. The substituents may be selected, for example, from C₁₋₆alkyl, C₂₋₆alkenyl, C₂₋₆alkynyl, C₃₋₈cycloalkyl, hydroxyl, oxo, C₁₋₆alkoxy, aryloxy, C₁₋₆alkoxyaryl, halo, C₁₋₆alkylhalo (such as CF₃ and CHF₂), C₁₋₆alkoxyhalo (such as OCF₃ and OCHF₂), carboxyl, esters, cyano, nitro, amino, substituted amino, disubstituted amino, acyl, ketones, amides, aminoacyl, substituted amides, disubstituted amides, thiol, alkylthio, thioxo, sulfates, sulfonates, sulfinyl, substituted sulfinyl, sulfonyl, substituted sulfonyl, sulfonylamides, substituted sulfonamides, disubstituted sulfonamides, aryl, arC₁₋₆alkyl, heterocyclyl and heteroaryl wherein each alkyl, alkenyl, alkynyl, cycloalkyl, aryl and heterocyclyl and groups containing them may be further optionally substituted. Optional substituents in the case N-heterocycles may also include but are not limited to C₁₋₆alkyl i.e. N—C₁₋₃alkyl, more preferably methyl particularly N-methyl.

A “therapeutically effective amount” refers to a quantity of a specified agent sufficient to achieve a desired effect in a subject being treated with that agent. Ideally, a therapeutically effective amount of an agent is an amount sufficient to inhibit or treat the disease or condition without causing a substantial cytotoxic effect in the subject. The therapeutically effective amount of an agent will be dependent on the subject being treated, the severity of the affliction, and the manner of administration of the therapeutic composition.

“Treatment” refers to a therapeutic intervention that ameliorates a sign or symptom of a disease or pathological condition after it has begun to develop. As used herein, the term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. The phrase “treating a disease” refers to inhibiting the full development of a disease, for example, in a subject who is at risk for a disease. A “prophylactic” treatment is a treatment administered to a subject who does not exhibit signs of a disease or exhibits only early signs for the purpose of decreasing the risk of developing a pathology or condition, or diminishing the severity of a pathology or condition.

“Pharmaceutical compositions” are compositions that include an amount (for example, a unit dosage) of one or more of the disclosed compounds together with one or more non-toxic pharmaceutically acceptable additives, including carriers, diluents, and/or adjuvants, and optionally other biologically active ingredients. Such pharmaceutical compositions can be prepared by standard pharmaceutical formulation techniques such as those disclosed in Remington's Pharmaceutical Sciences, Mack Publishing Co., Easton, PA (19th Edition).

The terms “pharmaceutically acceptable salt or ester” refers to salts or esters prepared by conventional means that include salts, e.g., of inorganic and organic acids, including but not limited to hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid and the like. “Pharmaceutically acceptable salts” of the presently disclosed compounds also include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylenediamine, chloroprocaine, diethanolamine, procaine, N-benzylphenethylamine, diethylamine, piperazine, tris (hydroxymethyl) aminomethane, and tetramethylammonium hydroxide. These salts may be prepared by standard procedures, for example by reacting the free acid with a suitable organic or inorganic base. Any chemical compound recited in this specification may alternatively be administered as a pharmaceutically acceptable salt thereof. “Pharmaceutically acceptable salts” are also inclusive of the free acid, base, and zwitterionic forms. Descriptions of suitable pharmaceutically acceptable salts can be found in Handbook of Pharmaceutical Salts, Properties, Selection and Use, Wiley VCH (2002). When compounds disclosed herein include an acidic function such as a carboxy group, then suitable pharmaceutically acceptable cation pairs for the carboxy group are well known to those skilled in the art and include alkaline, alkaline earth, ammonium, quaternary ammonium cations and the like. Such salts are known to those of skill in the art. For additional examples of “pharmacologically acceptable salts,” see Berge et al., J. Pharm. Sci. 66:1 (1977).

“Pharmaceutically acceptable esters” includes those derived from compounds described herein that are modified to include a carboxyl group. An in vivo hydrolysable ester is an ester, which is hydrolysed in the human or animal body to produce the parent acid or alcohol. Representative esters thus include carboxylic acid esters in which the non-carbonyl moiety of the carboxylic acid portion of the ester grouping is selected from straight or branched chain alkyl (for example, methyl, n-propyl, t-butyl, or n-butyl), cycloalkyl, alkoxyalkyl (for example, methoxymethyl), aralkyl (for example benzyl), aryloxyalkyl (for example, phenoxymethyl), aryl (for example, phenyl, optionally substituted by, for example, halogen, C.sub.1-4 alkyl, or C.sub.1-4 alkoxy) or amino); sulphonate esters, such as alkyl-or aralkylsulphonyl (for example, methanesulphonyl); or amino acid esters (for example, L-valyl or L-isoleucyl). A “pharmaceutically acceptable ester” also includes inorganic esters such as mono-, di-, or tri-phosphate esters. In such esters, unless otherwise specified, any alkyl moiety present advantageously contains from 1 to 18 carbon atoms, particularly from 1 to 6 carbon atoms, more particularly from 1 to 4 carbon atoms. Any cycloalkyl moiety present in such esters advantageously contains from 3 to 6 carbon atoms. Any aryl moiety present in such esters advantageously comprises a phenyl group, optionally substituted as shown in the definition of carbocycylyl above. Pharmaceutically acceptable esters thus include C₁-C₂₂ fatty acid esters, such as acetyl, t-butyl or long chain straight or branched unsaturated or omega-6 monounsaturated fatty acids such as palmoyl, stearoyl and the like. Alternative aryl or heteroaryl esters include benzoyl, pyridylmethyloyl and the like any of which may be substituted, as defined in carbocyclyl above. Additional pharmaceutically acceptable esters include aliphatic L-amino acid esters such as leucyl, isoleucyl and especially valyl. For therapeutic use, salts of the compounds are those wherein the counter-ion is pharmaceutically acceptable. However, salts of acids and bases which are non-pharmaceutically acceptable may also find use, for example, in the preparation or purification of a pharmaceutically acceptable compound.

The pharmaceutically acceptable acid and base addition salts as mentioned hereinabove are meant to comprise the therapeutically active non-toxic acid and base addition salt forms which the compounds are able to form. The pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic (i.e. hydroxybutanedioic acid), tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form.

The compounds containing an acidic proton may also be converted into their non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like.

The term “addition salt” as used hereinabove also comprises the solvates which the compounds described herein are able to form. Such solvates are for example hydrates, alcoholates and the like.

The term “quaternary amine” as used hereinbefore defines the quaternary ammonium salts which the compounds are able to form by reaction between a basic nitrogen of a compound and an appropriate quaternizing agent, such as, for example, an optionally substituted alkylhalide, arylhalide or arylalkylhalide, e.g. methyliodide or benzyliodide. Other reactants with good leaving groups may also be used, such as alkyl trifluoromethanesulfonates, alkyl methanesulfonates, and alkyl p-toluenesulfonates. A quaternary amine has a positively charged nitrogen. Pharmaceutically acceptable counterions include chloro, bromo, iodo, trifluoroacetate and acetate. The counterion of choice can be introduced using ion exchange resins.

Prodrugs of the disclosed compounds also are contemplated herein. A prodrug is an active or inactive compound that is modified chemically through in vivo physiological action, such as hydrolysis, metabolism and the like, into an active compound following administration of the prodrug to a subject. The term “prodrug” as used throughout this text means the pharmacologically acceptable derivatives such as esters, amides and phosphates, such that the resulting in vivo biotransformation product of the derivative is the active drug as defined in the compounds described herein. Prodrugs preferably have excellent aqueous solubility, increased bioavailability and are readily metabolized into the active inhibitors in vivo. Prodrugs of a compounds described herein may be prepared by modifying functional groups present in the compound in such a way that the modifications are cleaved, either by routine manipulation or in vivo, to the parent compound. The suitability and techniques involved in making and using prodrugs are well known by those skilled in the art. For a general discussion of prodrugs involving esters see Svensson and Tunek, Drug Metabolism Reviews 165 (1988) and Bundgaard, Design of Prodrugs, Elsevier (1985).

The term “prodrug” also is intended to include any covalently bonded carriers that release an active parent drug of the present invention in vivo when the prodrug is administered to a subject. Since prodrugs often have enhanced properties relative to the active agent pharmaceutical, such as, solubility and bioavailability, the compounds disclosed herein can be delivered in prodrug form. Thus, also contemplated are prodrugs of the presently disclosed compounds, methods of delivering prodrugs and compositions containing such prodrugs. Prodrugs of the disclosed compounds typically are prepared by modifying one or more functional groups present in the compound in such a way that the modifications are cleaved, either in routine manipulation or in vivo, to yield the parent compound. Prodrugs may include compounds having a phosphonate, hydroxy, thio and/or amino group functionalized with any group that is cleaved in vivo to yield the corresponding amino, hydroxy, thio and/or phosphonate group, respectively. Examples of prodrugs can include, without limitation, compounds having an acylated amino group and/or a phosphonate ester or phosphonate amide group.

Protected derivatives of the disclosed compounds also are contemplated. A variety of suitable protecting groups for use with the disclosed compounds are disclosed in Greene and Wuts, Protective Groups in Organic Synthesis; 3rd Ed.; John Wiley & Sons, New York, 1999.

In general, protecting groups are removed under conditions that will not affect the remaining portion of the molecule. These methods are well known in the art and include acid hydrolysis, hydrogenolysis and the like. One preferred method involves the removal of an ester, such as cleavage of a phosphonate ester using Lewis acidic conditions, such as in TMS-Br mediated ester cleavage to yield the free phosphonate. A second preferred method involves removal of a protecting group, such as removal of a benzyl group by hydrogenolysis utilizing palladium on carbon in a suitable solvent system such as an alcohol, acetic acid, and the like or mixtures thereof. A t-butoxy-based group, including t-butoxy carbonyl protecting groups can be removed utilizing an inorganic or organic acid, such as HCl or trifluoroacetic acid, in a suitable solvent system, such as water, dioxane and/or methylene chloride. Another exemplary protecting group, suitable for protecting amino and hydroxy functions amino is trityl. Other conventional protecting groups are known and suitable protecting groups can be selected by those of skill in the art in consultation with Greene and Wuts, Protective Groups in Organic Synthesis; 3rd Ed.; John Wiley & Sons, New York, 1999. When an amine is deprotected, the resulting salt can readily be neutralized to yield the free amine. Similarly, when an acid moiety, such as a phosphonic acid moiety is unveiled, the compound may be isolated as the acid compound or as a salt thereof.

Particular examples of the presently disclosed compounds may include one or more asymmetric centers; thus the compounds described can exist in different stereoisomeric forms. Accordingly, compounds and compositions may be provided as individual pure enantiomers or as stereoisomeric mixtures, including racemic mixtures. In certain embodiments the compounds disclosed herein may be synthesized in or may be purified to be in substantially enantiopure form, such as in a 90% enantiomeric excess, a 95% enantiomeric excess, a 97% enantiomeric excess or even in greater than a 99% enantiomeric excess, such as in enantiopure form.

The presently disclosed compounds can have at least one asymmetric center or geometric center, cis-trans center (C═C, C═N). All chiral, diasteromeric, racemic, meso, rotational and geometric isomers of the structures are intended unless otherwise specified. The compounds can be isolated as a single isomer or as mixture of isomers. All tautomers of the compounds are also considered part of the disclosure. The presently disclosed compounds also include all isotopes of atoms present in the compounds, which can include, but are not limited to, deuterium, tritium, ¹⁸F, etc.

### Compounds

Disclosed herein are compounds, or pharmaceutically acceptable salts thereof, of formula I:

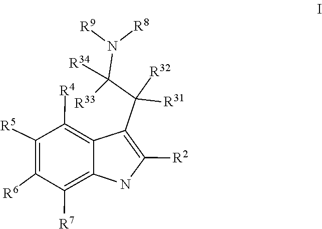

wherein R¹, R⁸, R⁹, R³¹, R³², R³³, and R³⁴ are each independently H, C₁-C₆ alkyl, or substituted C₁-C₆ alkyl; and
R², R⁴, R⁵, R⁶, and R⁷ are each independently H, F, Cl, Br, —OR¹, —NR⁸R⁹, C₁-C₆ alkyl, or substituted C₁-C₆ alkyl.

In certain embodiments of formula I, R⁸ and R⁹ are each H.

In certain embodiments of formula I, R⁸ and R⁹ are each independently C₁-C₆ alkyl.

In certain embodiments of formula I, at least one of R⁴, R⁵, R⁶, and R⁷ is F, Cl, or Br, and the remaining R⁴, R⁵, R⁶, and R⁷ are each H.

In certain embodiments of formula I, R⁴, R⁵, R⁶, or R⁷ is F.

In certain embodiments of formula I, R³¹, R³², R³³, and R³⁴ are each H.

In certain embodiments of formula I, the substituted C₁-C₆ alkyl is alkyl-substituted C₁-C₆ alkyl, aryl-substituted C₁-C₆ alkyl, or heteroaryl-substituted C₁-C₆ alkyl.

In certain embodiments of formula I, R¹ is heteroaryl-substituted C₁-C₆ alkyl, particularly N-heteroaryl-substituted C₁-C₆ alkyl, and more particularly N-heteroaryl-substituted C₁ alkyl.

Disclosed herein are compounds, or pharmaceutically acceptable salts thereof, of formula II:

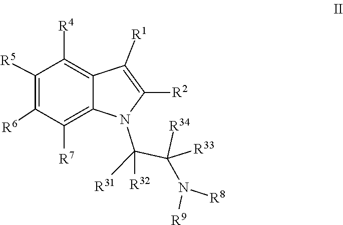

wherein R¹, R⁸, R⁹, R³¹, R³², R³³, and R³⁴ are each independently H, C₁-C₆ alkyl, or substituted C₁-C₆ alkyl; and
R², R⁴, R⁵, R⁶, and R⁷ are each independently H, F, Cl, Br, —OR¹, —NR⁸R⁹, C₁-C₆ alkyl, or substituted C₁-C₆ alkyl.

In certain embodiments of formula II, R⁸ and R⁹ are each H.

In certain embodiments of formula II, R⁸ and R⁹ are each independently C₁-C₆ alkyl.

In certain embodiments of formula II, at least one of R⁴, R⁵, R⁶, and R⁷ is F, Cl, or Br, and the remaining R⁴, R⁵, R⁶, and R⁷ are each H.

In certain embodiments of formula II, R⁴, R⁵, R⁶, or R⁷ is F.

In certain embodiments of formula II, R³¹, R³², R³³, and R³⁴ are each H.

In certain embodiments of formula II, the substituted C₁-C₆ alkyl is alkyl-substituted C₁-C₆ alkyl, aryl-substituted C₁-C₆ alkyl, or heteroaryl-substituted C₁-C₆ alkyl.

In certain embodiments of formula II, R¹ is heteroaryl-substituted C₁-C₆ alkyl, particularly N-heteroaryl-substituted C₁-C₆ alkyl, and more particularly N-heteroaryl-substituted C₁ alkyl.

Disclosed herein are compounds, or pharmaceutically acceptable salts thereof, of formula III:

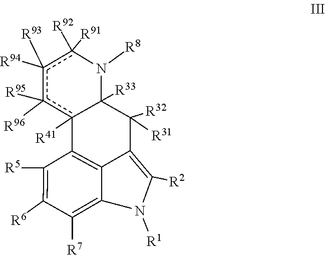

wherein R¹ and R⁸ are each independently H, C₁-C₆ alkyl, or substituted C₁-C₆ alkyl;
R², R³, R⁶, R⁷, R⁴¹, R⁹³, R⁹⁴, R⁹⁵, and R⁹⁶ are each independently H, F, Cl, Br, —OR¹, —NR⁸R⁹, —CR¹R¹OR¹, —CO₂R¹, —CONR⁸R⁹, C₁-C₆ alkyl, or substituted C₁-C₆ alkyl;
R⁹, R³¹, R³², R³³, R⁹¹, and R⁹² are each independently H, —CR¹R¹OR¹, —CO₂R¹, —CONR⁸R⁹, C₁-C₆ alkyl, or substituted C₁-C₆ alkyl; and
where each bond represented by [symbol] is a single or double bond as needed to satisfy valence requirements, and where each of R⁴¹, R⁹⁵, R⁹⁴, and R⁹¹ may or may not be present as needed to satisfy valency requirements.

In certain embodiments of formula III, the compound is of formula IIIa

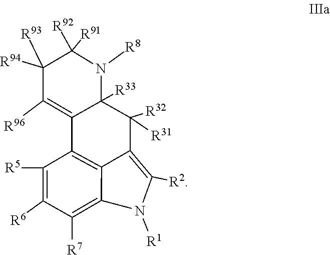

In certain embodiments of formula III, the compound is of formula IIIb

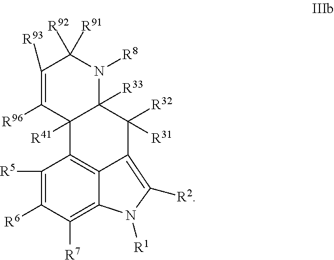

In certain embodiments of formula III, the compound is of formula IIIc

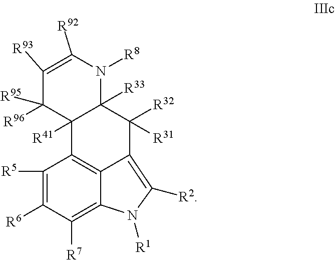

In certain embodiments of formula III, the compound is of formula IIId

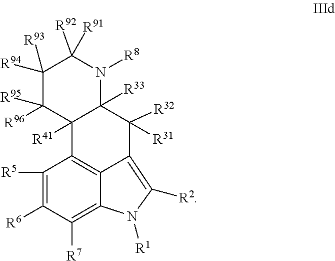

In certain embodiments of formula III, R¹ is H.

In certain embodiments of formula III, R⁸ is methyl.

In certain embodiments of formula III, R², R³, R⁶, R⁷, R³¹, R³², R³³, R⁴¹, R⁹¹, and R⁹² are each H.

In certain embodiments of formula III, R⁹³ is —CR¹R¹OR¹, wherein each R¹ is H, and R⁹⁴ is H.

Disclosed herein are compounds, or pharmaceutically acceptable salts thereof, of formula IV:

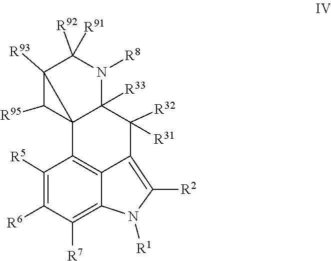

wherein R¹ and R⁸ are each independently H, C₁-C₆ alkyl, or substituted C₁-C₆ alkyl;
R², R⁵, R⁶, R⁷, R⁹³, and R⁹⁵ are each independently H, F, Cl, Br, —OR¹, —NR⁸R⁹, —CR¹R¹OR¹, —CO₂R¹, —CONR⁸R⁹, C₁-C₆ alkyl, or substituted C₁-C₆ alkyl; and
R⁹, R³¹, R³², R³³, R⁹¹, and R⁹² are each independently H, —CR¹R¹OR¹, —CO₂R¹, —CONR⁸R⁹, C₁-C₆ alkyl, or substituted C₁-C₆ alkyl.

In certain embodiments of formula IV, R¹ is H.

In certain embodiments of formula IV, R⁸ is methyl.

In certain embodiments of formula IV, R², R⁵, R⁶, R⁷, R³¹, R³², R³³, R⁴¹, R⁹¹, and R⁹² are each H.

Disclosed herein are compounds, or pharmaceutically acceptable salts thereof, of formula V:

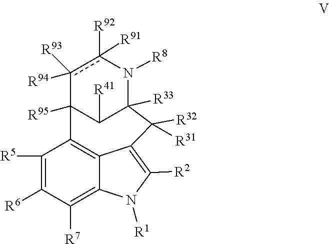

wherein R¹ and R⁸ are each independently H, C₁-C₆ alkyl, or substituted C₁-C₆ alkyl;
R², R⁵, R⁶, R⁷, R⁴¹, R⁹³, R⁹⁴ and R⁹⁵ are each independently H, F, Cl, Br, —OR¹, —NR⁸R⁹, —CR¹R¹OR¹, —CO₂R¹, —CONR⁸R⁹, C₁-C₆ alkyl, or substituted C₁-C₆ alkyl; a
R⁹, R³¹, R³², R³³, R⁹¹, and R⁹² are each independently H, —CR¹R¹OR¹, —CO₂R¹, —CONR⁸R⁹, C₁-C₆ alkyl, or substituted C₁-C₆ alkyl; and
where each bond represented by [symbol] is a single or double bond as needed to satisfy valence requirements, and where each of R⁹⁴ and R⁹¹ may or may not be present as needed to satisfy valency requirements.

In certain embodiments of formula V, R¹ is H.

In certain embodiments of formula V, R⁸ is methyl.

In certain embodiments of formula V, R², R⁵, R⁶, R⁷, R³¹, R³², R³³, R⁴¹, R⁹¹, R⁹², and R⁹⁵ are each H.

In certain embodiments of formula V, R⁹³ is —CONR⁸R⁹, wherein R⁸ and R⁹ are each C₁-C₆ alkyl, and R⁹⁴ is H.

### Pharmaceutical Compositions and Methods of Use

In certain embodiments, the compounds disclosed herein are psychedelic agents.

In certain embodiments, the compounds disclosed herein have selective affinities to serotonin receptors, specifically 5-HT, and a diverse profile of binding to dopamine receptors. The psychoactive effects are mainly attributed to 5-HT2A and 5-HT2C agonism, while behavioral effects are linked to dopamine D2 receptor activity.

In certain embodiments, the compounds disclosed herein are 5-HT agonists, particularly 5-HT2A and 5-HT2C agonists. A “5-HT agonist” refers to a compound that increases the activity of a 5-hydroxytryptamine receptor.

The compounds disclosed herein may be used to improve the well-being of a subject. As used herein, “well-being” refers to a positive state of health or comfort, e.g., relative to a reference population. As used herein “mental well-being” refers to a positive mental state, relative to a reference population. For example, in an individual having depression, low self-esteem, addiction, compulsion, or anxiety may experience an improvement in mental well-being in response to therapy aimed at improving mood, self-esteem, addiction, compulsion, or anxiety, respectively. As used herein, “physical well-being” refers to one or more positive aspects of an individual's physical health. For example, an improvement of physical well-being includes alleviation of somatic symptoms associated with a psychological disorder, depression, addiction, compulsion, anxiety, or sexual dysfunction. Such symptoms include, for example, chronic pain, pain disorder, relational disorder, body dysmorphia, conversion (e.g., loss of bodily function due to anxiety), hysteria, neurological conditions without identifiable cause, or psychosomatic illness).

The compounds disclosed herein may also be used for treating a neurological condition in a subject. The neurological condition may be depression, memory loss, dementia, cognitive dysfunction, hearing loss, vision loss, neurologic pain, psychological disorder, or combinations thereof.

As used herein, a “psychological disorder” refers to a condition characterized by a disturbance in one's emotional or behavioral regulation that reflects a dysfunction in the psychological, biological, or developmental processes underlying mental function. Psychological disorders include, but are not limited to depressive disorders (major depression, melancholic depression, atypical depression, or dysthymia), anxiety disorders (end of life anxiety, generalized anxiety disorder, panic disorder, social anxiety, post-traumatic stress disorder, acute stress disorder, obsessive compulsive disorder, or social phobia), addictions (e.g., substance abuse, e.g., alcohol, tobacco, or drug abuse)), and compulsive behavior disorders (e.g., primary impulse-control disorders or obsessive-compulsive disorder). Psychological disorders can be any psychological condition associated with one or more symptoms, e.g., somatic symptoms (e.g., chronic pain, anxiety disproportionate to severity of physical complaints, pain disorder, body dysmorphia, conversion (i.e., loss of bodily function due to anxiety), hysteria, or neurological conditions without identifiable cause), or psychosomatic symptoms. Psychological disorders also include repetitive body-focused behaviors, such as tic disorders (e.g., Tourette's Syndrome, trichotillomania, nail-biting, temporomandibular disorder, thumb-sucking, repetitive oral-digital, lip-biting, fingernail biting, eye-rubbing, skin-picking, or a chronic motor tic disorder). In some cases, development of a psychological disorder is associated with or characterized by a prodromal symptom, such as depressed mood, decreased appetite, weight loss, increased appetite, weight gain, initial insomnia, middle insomnia, early waking, hypersomnia, decreased energy, decreased interest or pleasure, self-blame, decreased concentration, indecision, suicidality, psychomotor agitation, psychomotor retardation, crying more frequently, inability to cry, hopelessness, worrying/brooding, decreased self-esteem, irritability, dependency, self-pity, somatic complaints, decreased effectiveness, helplessness, and decreased initiation of voluntary responses.

In some embodiments, the methods disclosed herein involve administering to a subject in need of treatment a pharmaceutical composition, for example a composition that includes a pharmaceutically acceptable carrier and a therapeutically effective amount of one or more of the compounds disclosed herein. The compounds may be administered orally, parenterally (including subcutaneous injections (SC or depo-SC), intravenous (IV), intramuscular (IM or depo-IM), intrasternal injection or infusion techniques), sublingually, intranasally (inhalation), intrathecally, topically, ophthalmically, or rectally. The pharmaceutical composition may be administered in dosage unit formulations containing conventional non-toxic pharmaceutically acceptable carriers, adjuvants, and/or vehicles. The compounds are preferably formulated into suitable pharmaceutical preparations such as tablets, capsules, or elixirs for oral administration or in sterile solutions or suspensions for parenteral administration. Typically the compounds described above are formulated into pharmaceutical compositions using techniques and procedures well known in the art.

In some embodiments, one or more of the disclosed compounds are mixed or combined with a suitable pharmaceutically acceptable carrier to prepare a pharmaceutical composition. Pharmaceutical carriers or vehicles suitable for administration of the compounds provided herein include any such carriers known to be suitable for the particular mode of administration. Remington: The Science and Practice of Pharmacy, The University of the Sciences in Philadelphia, Editor, Lippincott, Williams, & Wilkins, Philadelphia, PA, 21^{st} Edition (2005), describes exemplary compositions and formulations suitable for pharmaceutical delivery of the compounds disclosed herein. In addition, the compounds may be formulated as the sole pharmaceutically active ingredient in the composition or may be combined with other active ingredients.

Upon mixing or addition of the compound(s) to a pharmaceutically acceptable carrier, the resulting mixture may be a solution, suspension, emulsion, or the like. Liposomal suspensions may also be suitable as pharmaceutically acceptable carriers. These may be prepared according to methods known to those skilled in the art. The form of the resulting mixture depends upon a number of factors, including the intended mode of administration and the solubility of the compound in the selected carrier or vehicle. Where the compounds exhibit insufficient solubility, methods for solubilizing may be used. Such methods are known and include, but are not limited to, using cosolvents such as dimethylsulfoxide (DMSO), using surfactants such as Tween®, and dissolution in aqueous sodium bicarbonate. Derivatives of the compounds, such as salts or prodrugs may also be used in formulating effective pharmaceutical compositions. The disclosed compounds may also be prepared with carriers that protect them against rapid elimination from the body, such as time-release formulations or coatings. Such carriers include controlled release formulations, such as, but not limited to, microencapsulated delivery systems.

The active compound is included in the pharmaceutically acceptable carrier in an amount sufficient to exert a therapeutically useful effect in the absence of undesirable side effects on the subject treated. A therapeutically effective concentration may be determined empirically by testing the compounds in known in vitro and in vivo model systems for the treated disorder. In some examples, a therapeutically effective amount of the compound is an amount that lessens or ameliorates at least one symptom of the disorder for which the compound is administered. Typically, the compositions are formulated for single dosage administration. The concentration of active compound in the drug composition will depend on absorption, inactivation, and excretion rates of the active compound, the dosage schedule, and amount administered as well as other factors known to those of skill in the art.

In some examples, about 0.1 mg to 1000 mg of a disclosed compound, a mixture of such compounds, or a physiologically acceptable salt or ester thereof, is compounded with a physiologically acceptable vehicle, carrier, excipient, binder, preservative, stabilizer, flavor, etc., in a unit dosage form. The amount of active substance in those compositions or preparations is such that a suitable dosage in the range indicated is obtained. The term “unit dosage form” refers to physically discrete units suitable as unitary dosages for human subjects and other mammals, each unit containing a predetermined quantity of active material calculated to produce the desired therapeutic effect, in association with a suitable pharmaceutical excipient. In some examples, the compositions are formulated in a unit dosage form, each dosage containing from about 1 mg to about 1000 mg (for example, about 2 mg to about 500 mg, about 5 mg to 50 mg, about 10 mg to 100 mg, or about 25 mg to 75 mg) of the one or more compounds. In other examples, the unit dosage form includes about 0.1 mg, about 1 mg, about 5 mg, about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 400 mg, about 500 mg, about 600 mg, about 700 mg, about 800 mg, about 900 mg, about 1000 mg, or more of the disclosed compound(s).

The disclosed compounds or compositions may be administered as a single dose, or may be divided into a number of smaller doses to be administered at intervals of time. The therapeutic compositions can be administered in a single dose delivery, by continuous delivery over an extended time period, in a repeated administration protocol (for example, by a multi-daily, daily, weekly, or monthly repeated administration protocol). It is understood that the precise dosage, timing, and duration of treatment is a function of the disease being treated and may be determined empirically using known testing protocols or by extrapolation from in vivo or in vitro test data. It is to be noted that concentrations and dosage values may also vary with the severity of the condition to be alleviated. In addition, it is understood that for a specific subject, dosage regimens may be adjusted over time according to the individual need and the professional judgment of the person administering or supervising the administration of the compositions, and that the concentration ranges set forth herein are exemplary only.

When administered orally as a suspension, these compositions are prepared according to techniques well known in the art of pharmaceutical formulation and may contain microcrystalline cellulose for imparting bulk, alginic acid or sodium alginate as a suspending agent, methylcellulose as a viscosity enhancer, and sweeteners/flavoring agents. As immediate release tablets, these compositions may contain microcrystalline cellulose, dicalcium phosphate, starch, magnesium stearate and lactose and/or other excipients, binders, extenders, disintegrants, diluents and lubricants. If oral administration is desired, the compound is typically provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules, or troches. Pharmaceutically compatible binding agents and adjuvant materials can be included as part of the composition. The tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a gildant, such as, but not limited to, colloidal silicon dioxide; a sweetening agent such as sucrose or saccharin; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings, and flavors.

When administered orally, the compounds can be administered in usual dosage forms for oral administration. These dosage forms include the usual solid unit dosage forms of tablets and capsules as well as liquid dosage forms such as solutions, suspensions, and elixirs. When the solid dosage forms are used, it is preferred that they be of the sustained release type so that the compounds need to be administered only once or twice daily. In some examples, an oral dosage form is administered to the subject 1, 2, 3, 4, or more times daily. In additional examples, the compounds can be administered orally to humans in a dosage range of 1 to 1000 mg/kg body weight in single or divided doses. One illustrative dosage range is 0.1 to 200 mg/kg body weight orally (such as 0.5 to 100 mg/kg body weight orally) in single or divided doses. For oral administration, the compositions may be provided in the form of tablets containing about 1 to 1000 milligrams of the active ingredient, particularly 1, 5, 10, 15, 20, 25, 50, 75, 100, 150, 200, 250, 300, 400, 500, 600, 750, 800, 900, or 1000 milligrams of the active ingredient. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Injectable solutions or suspensions may also be formulated, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono-or diglycerides, and fatty acids, including oleic acid. Solutions or suspensions used for parenteral, intradermal, subcutaneous, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerine, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers.

The compounds can be administered parenterally, for example, by IV, IM, depo-IM, SC, or depo-SC. When administered parenterally, a therapeutically effective amount of about 0.1 to about 500 mg/day (such as about 1 mg/day to about 100 mg/day, or about 5 mg/day to about 50 mg/day) may be delivered. When a depot formulation is used for injection once a month or once every two weeks, the dose may be about 0.1 mg/day to about 100 mg/day, or a monthly dose of from about 3 mg to about 3000 mg.

The compounds can also be administered sublingually. When given sublingually, the compounds should be given one to four times daily in the amounts described above for IM administration.

The compounds can also be administered intranasally. When given by this route, the appropriate dosage forms are a nasal spray or dry powder. The dosage of the compounds for intranasal administration is the amount described above for IM administration. When administered by nasal aerosol or inhalation, these compositions may be prepared according to techniques well known in the art of pharmaceutical formulation and may be prepared as solutions in saline, employing benzyl alcohol or other suitable preservatives, absorption promoters to enhance bioavailability, fluorocarbons, and/or other solubilizing or dispersing agents.

The compounds can be administered intrathecally. When given by this route, the appropriate dosage form can be a parenteral dosage form. The dosage of the compounds for intrathecal administration is the amount described above for IM administration.

The compounds can be administered topically. When given by this route, the appropriate dosage form is a cream, ointment, or patch. When administered topically, an illustrative dosage is from about 0.5 mg/day to about 200 mg/day. Because the amount that can be delivered by a patch is limited, two or more patches may be used.

The compounds can be administered rectally by suppository. When administered by suppository, an illustrative therapeutically effective amount may range from about 0.5 mg to about 500 mg. When rectally administered in the form of suppositories, these compositions may be prepared by mixing the drug with a suitable non-irritating excipient, such as cocoa butter, synthetic glyceride esters of polyethylene glycols, which are solid at ordinary temperatures, but liquefy and/or dissolve in the rectal cavity to release the drug.

It should be apparent to one skilled in the art that the exact dosage and frequency of administration will depend on the particular compounds administered, the particular condition being treated, the severity of the condition being treated, the age, weight, general physical condition of the particular subject, and other medication the individual may be taking as is well known to administering physicians or other clinicians who are skilled in therapy of retroviral infections, diseases, and associated disorders.

### EXAMPLES

### Assays

TABLE 1
Percent Inhibition of the Binding of a Specific Radioligand at Serotonin Receptors by Lysergols
and Isolysergols^{[a]}

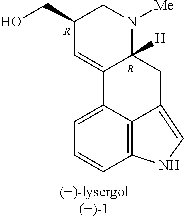

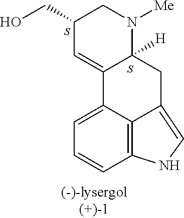

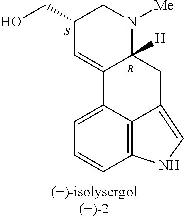

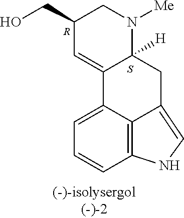

| cmpd | 5-HT_{1A} | 5-HT_{2A} | 5-HT_{2B} | 5-HT_{2C} |
|---|---|---|---|---|
| (+)-1 | 94^{[b]}, 99^{[c]} | 96^{[b]}, 97^{[c]} | 96^{[b]}, 99^{[c]} | 97^{[b]}, 99^{[c]} |
| (−)-1 | 27^{[b]}, 67^{[c]} | 12^{[b]}, 62^{[c]} | 34^{[b]}, 87^{[c]} | 41^{[b]}, 86^{[c]} |
| (+)-2 | 96^{[b]}, 99^{[c]} | 92^{[b]}, 99^{[c]} | 90^{[b]}, 93^{[c]} | 91^{[b]}, 97^{[c]} |
| (−)-2 | 9^{[b]}, 19^{[c]} | 0^{[b]}, 20^{[c]} | 3^{[b]}, 16^{[c]} | 5^{[b]}, 44^{[c]} |

^{[a]}Biochemical assays were performed in duplicate at human receptors at Eurofins Cerep Panlabs;
^{[b]}% inh. at 100 nM;
^{[c]}% inh. at 1.0 μM

TABLE 2
Effects of cycloclavines and D-LSD Expressed as Percent
Inhibition [% inh] of Specific Binding of a Radioligand
Standard to Selected CNS Receptors^{a}

| | | (+)- | (+)- | (−)- | (−)- | |
|---|---|---|---|---|---|---|
| | | Cyclo- | Cyclo- | Cyclo- | Cyclo- | D-LSD |
| | | clavine | clavine | clavine | clavine | [10 |
| Entry | Receptor | [10 μM] | [1 μM] | [10 μM] | [1 μM] | μM]^{c} |
| 1^{b} | Adrenergic α₁ | 57 | | 59 | | 100^{d} |
| 2 | Dopamine D₁ | 91 | | 30 | | 93 |
| 3 | Dopamine D_{2L} | 81 | | 14 | | 85 |
| 4 | Dopamine D₃ | 93 | 75 | 64 | 32 | 78 |
| 5^{b} | GABA_{A} | 9 | | 0 | | 0 |
| 6 | Histamine H₁ | 17 | | 27 | | 85^{b} |
| 7 | Muscarinic M₂ | 0 | | 0 | | 2 |
| 8 | Muscarinic M₅ | 11 | | 15 | | 15 |
| 9 | Nicotinic Acetyl- | 9 | | 11 | | 0 |
| | choline α4β2 | | | | | |
| 10 | Opiate κ | 73 | | 75 | | 29 |
| 11 | Orexin OX₁ | 23 | | 6 | | ND |
| 12 | Serotonin 5-HT_{1A} | 97 | 97 | 91 | 73 | 100 |
| 13 | Serotonin 5-HT_{2A} | 100 | 100 | 89 | 51 | 93 |

^{a}Biochemical assays were performed in duplicate at human receptors at Eurofins Cerep Panlabs and are presented as the percent inhibition of specific binding or activity of a radioligand, unless otherwise indicated;
^{b}rat receptor data;
^{c}data from ref. 34;
^{d}data from ref. 35.
ND = not determined.

TABLE 3
Effects of Cycloclavines, DMT, Psilocin, and D-
LSD on 5-HY Subtypes and Sigma-1 Receptors^{a, b}

| | | (+)- | (−)- | | | |
|---|---|---|---|---|---|---|
| | | Cyclo- | Cyclo- | | | |
| | | clavine | clavine | DMT^{c} | Psilocin^{c} | D-LSD^{c} |
| Entry | Receptor | [μM] | [μM] | [μM] | [μM] | [μM] |
| 1 | Serotonin 5- | 0.14 | ~5 | 0.075^{d} | 0.123^{d} | 0.003^{d} |
| | HT_{1A} | | | | | |
| 2 | Serotonin 5- | ~10 | >50 | 0.076 | 0.721 | 0.261 |
| | HT_{2A} | | | | | |
| 3 | Serotonin 5- | >20 | >20 | 3.4 | >20 | 12 |
| | HT_{2B} | | | | | |
| 4 | Serotonin 5- | 0.016 | 3.2 | 0.424^{d} | 0.094^{d} | 0.015^{d} |
| | HT_{2C} | | | | | |
| 5^{d} | Sigma-1 | ~50 | 8.3 | 5.2^{e} | >10^{e} | ND |

^{a}Biochemical assays were performed in duplicate at human receptors at Eurofins Cerep Panlabs and results are based on 5-point concentration response curves, unless otherwise indicated;
^{b}activation potency EC₅₀ values are shown, unless otherwise specified;
^{c}data from ref. 36, unless otherwise specified;
^{d}inhibition constants Kᵢ;
^{e}data from ref. 37.
ND = not determined.

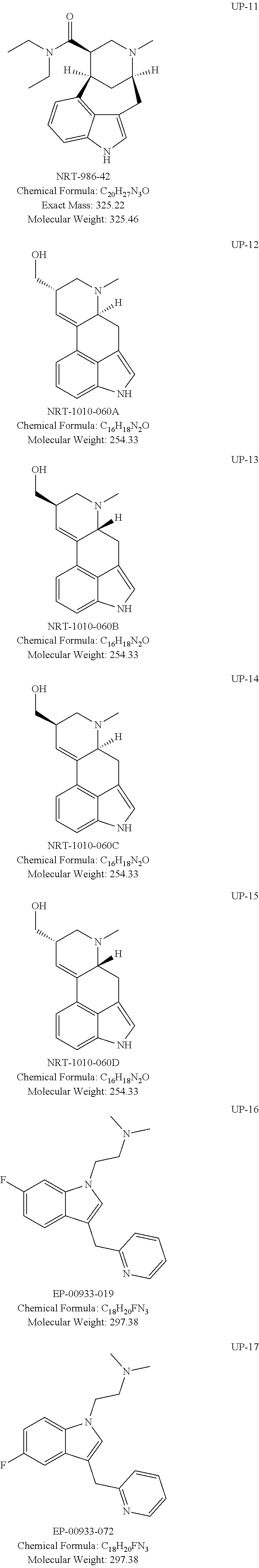

### Experimentals

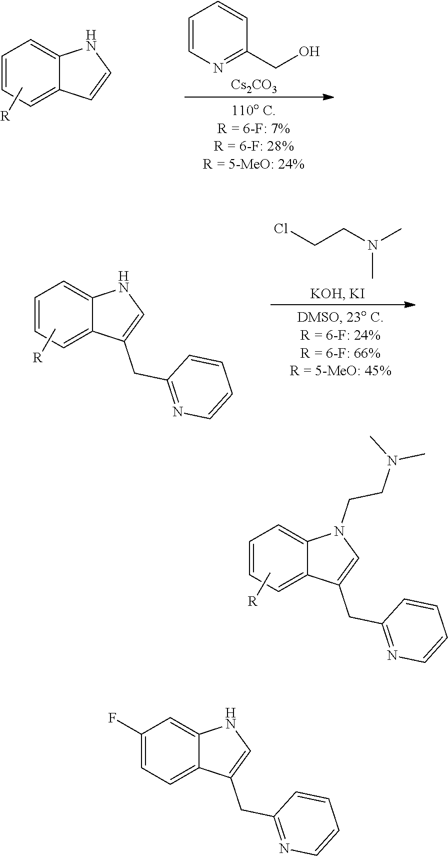

6-fluoro-3-(pyridin-2-ylmethyl)-1H-indole: A mixture of 6-fluoroindole (250 mg. 1.81 mmol). Cs₂CO₃ (656 mg, 1.99 mmol) and 2-pyridinemethanol (0.535 mL, 5.35 mmol) was added to a 10 mL microwave vial. The vial was sealed, and the mixture was heated and stirred at 110° C. for 50 hours. The mixture was cooled to room temperature and diluted with CH₂Cl₂ (20 mL) and H₂O (10 mL). The aqueous layer was extracted with 9:1 CH₂Cl₂:MeOH (3×10 mL) and the combined organic layers were washed with brine (30 mL), dried (MgSO₄), filtered, and concentrated. Purification by flash column chromatography on SiO₂ (35% EtOAc/CH₂Cl₂) afforded 6-fluoro-3-(pyridin-2-ylmethyl)-1H-indole (30.1 mg, 0.133 mmol, 7%) as a tan-brown solid: ¹H NMR (400 MHz, CDCl₃) δ 8.55 (d, J=4.9 Hz, 1H), 8.18 (bs, 1H), 7.56 (td, J=1.8, 7.7 Hz, 1H), 7.40 (dd, J=5.3, 8.6 Hz, 1H), 7.17 (d, J=7.8, 1H), 7.12 (dd, J=5.6, 7.1 Hz, 1H), 7.04-7.00 (m, 2H), 6.82 (td, J=2.3, 9.6 Hz, 1H), 4.28 (s, 2H).

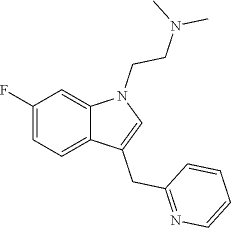 [SMILES: CN(C)CCn1cc(Cc2ccccn2)c2ccc(F)cc21]

2-(6-fluoro-3-(pyridin-2-ylmethyl)-1H-indol-1-yl)-N,N-dimethylethan-1-amine: To a solution of 6-fluoro-3-(pyridin-2-ylmethyl)-1H-indole (30.1 mg, 0.133 mmol) in DMSO (0.33 mL) was added 2-chloro-N,N-dimethylethylamine hydrochloride (21.2 mg, 0.146 mmol), potassium iodide (24.4 mg, 0.146 mmol), and potassium hydroxide (37.2 mg, 0.665 mmol). The reaction was stirred at room temperature for 24 hours before being diluted with aqueous NaOH (1 M, 10 mL). The aqueous phase was extracted with CH₂Cl₂ (3×15 mL), and the combined organic extracts were dried (MgSO₄), filtered, and concentrated. Purification by flash column chromatography on SiO₂ (10% MeOH/EtOAc w/1% Et₃N) afforded 2-(6-fluoro-3-(pyridin-2-ylmethyl)-1H-indol-1-yl)-N,N-dimethylethan-1-amine (9.40 mg, 0.0316 mmol, 24%) as a brown oil: IR (ATR) νₘₐₓ 3058, 2945, 2772, 1620, 1591, 1568, 1472, 1435, 1374, 1335, 1255, 1219, 1146, 1115, 1094, 1049, 1017, 995, 922, 895, 825, 800, 749, 707, 670 cm⁻¹; ¹H NMR (500 MHz, CDCl₃) δ 8.55 (d, J=4.5 Hz, 1H), 7.54 (td, J=1.5, 7.5 Hz, 1H), 7.38 (dd, J=5.5, 9 Hz, 1H), 7.14 (d, J=8 Hz, 1H), 7.10 (dd, J=5, 7 Hz, 1H), 6.99-6.97 (m with apparent singlet at 6.99, 2H), 6.80 (td, J=2, 8.5 Hz, 1H), 4.24 (s, 2H), 4.15 (t, J=7 Hz, 2H), 2.71 (t, J=7 Hz, 2H), 2.32 (s, 6H); ¹³C NMR (125 MHz, CDCl₃) δ 161.2, 161.0, 159.2, 149.3, 136.7, 136.6, 126.9 (d, J_{C,F}=3.5 Hz), 124.6, 122.0 (d, J_{C,F}=196 Hz, 120.4 (d, J_{C,F}=10 Hz), 113.2, 107.8 (d, J_{C,F}=24 Hz), 95.8 (d, J_{C,F}=26 Hz), 58.8, 45.8, 44.7, 34.6; HRMS (ESI+) m/z calculated for C₁₈H₂₀FN₃, [M+H]⁺ 298.17140, found 298.17049.

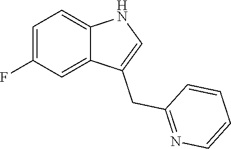 [SMILES: Fc1ccc2[nH]cc(Cc3ccccn3)c2c1]

5-fluoro-3-(pyridin-2-ylmethyl)-1H-indole: A mixture of 5-fluoroindole (138 mg, 1.00 mmol), Cs2CO3 (362 mg, 1.10 mmol) and 2-pyridinemethanol (0.89 mL, 9.00 mmol) was added to a 10 mL microwave vial. The vial was sealed, and the mixture was heated and stirred at 110° C. for 69 hours. The mixture was cooled to room temperature and diluted with CH₂Cl₂ (20 mL) and H₂O (10 mL). The aqueous layer was extracted with 9:1 CH₂Cl₂:MeOH (3×10 mL) and the combined organic layers were washed with brine (30 mL), dried (MgSO₄), filtered, and concentrated. Purification by flash column chromatography on SiO₂ (35% EtOAc/CH₂Cl₂) afforded 5-fluoro-3-(pyridin-2-ylmethyl)-1H-indole (63.1 mg, 0.279 mmol, 28%) as a tan-brown solid: ¹H NMR (400 MHz, CDCl₃) δ 8.56 (d, J=4.9 Hz, 1H), 8.03 (bs, 1H), 7.56 (td, J=1.8, 7.7 Hz, 1H), 7.28-7.25 (m, 1H), 7.17-7.10 (m, 4H), 6.92 (td, J=2.5, 9 Hz, 1H), 4.25 (s, 2H).

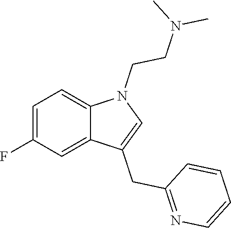 [SMILES: CN(C)CCn1cc(Cc2ccccn2)c2cc(F)ccc21]

2-(5-fluoro-3-(pyridin-2-ylmethyl)-1H-indol-1-yl)-N,N-dimethylethan-1-amine: To a solution of 5-fluoro-3-(pyridin-2-ylmethyl)-1H-indole (61.0 mg, 0.271 mmol) in DMSO (0.68 mL) was added 2-chloro-N,N-dimethylethylamine hydrochloride (43.3 mg, 0.298 mmol), potassium iodide (49.9 mg, 0.289 mmol), and potassium hydroxide (76.7 mg, 1.35 mmol). The reaction was stirred at room temperature for 24 hours before being diluted with aqueous NaOH (1 M, 10 mL). The aqueous phase was extracted with CH₂Cl₂ (3×15 mL), and the combined organic extracts were dried (MgSO₄), filtered, and concentrated. Purification by flash column chromatography on SiO₂ (10% MeOH/EtOAc w/1% Et₃N) afforded 2-(5-fluoro-3-(pyridin-2-ylmethyl)-1H-indol-1-yl)-N,N-dimethylethan-1-amine (52.9 mg, 0.178 mmol, 66%) as a brown oil: IR (ATR) νₘₐₓ 3059, 2942, 2770, 1623, 1591, 1569, 1486, 1453, 1434, 1377, 1358, 1306, 1252, 1228, 1205, 1149, 1094, 1048, 994, 907, 851, 789, 767, 749, 695 cm⁻¹; ¹H NMR (300 MHz, CDCl₃) δ 8.54 (d, J=4.8 Hz, 1H), 7.54 (td, J=1.8, 7.7 Hz, 1H), 7.23 (dd, J=4.3, 7.4 Hz, 1H), 7.15-7.07 (m, 4H), 6.92 (td, J=2.5, 9.1 Hz, 1H), 4.25-4.20 (m, 4H), 2.75 (t, J=7.2 Hz, 2H), 2.33 (s, 6H); ¹³C NMR (75 MHz, CDCl₃) δ 161.0, 159.3, 156.2.2, 149.3, 136.6, 133.1, 128.3 (d, J_{C,F}=9 Hz), 128.1, 122.0 (d, J_{C,F}=113 Hz, 112.9 (d, J_{C,F}=4.8 Hz), 110.1 (d, J_{C,F}=34.7 Hz), 110.0, 104.5 (d, J_{C,F}=23.2 Hz), 58.8, 45.6, 44.5, 34.5; HRMS (ESI+) m/z calculated for C₁₈H₂₀FN₃, [M+H]⁺ 298.17140, found 298.17084.

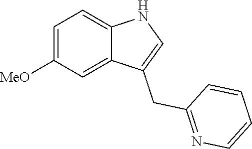 [SMILES: COc1ccc2[nH]cc(Cc3ccccn3)c2c1]

5-methoxy-3-(pyridin-2-ylmethyl)-1H-indole: A mixture of 5-methoxyindole (149 mg, 1.00 mmol), Cs2CO3 (363 mg, 1.1 mmol) and 2-pyridinemethanol (0.296 mL, 3.00 mmol) was added to a 10 mL microwave vial. The vial was sealed, and the mixture was heated and stirred at 110° C. for 91 hours. The mixture was cooled to room temperature and diluted with CH₂Cl₂ (20 mL) and H₂O (10 mL). The aqueous layer was extracted with 9:1 CH₂Cl₂:MeOH (3×10 mL), and the combined organic layers were washed with brine (30 mL), dried (MgSO₄), filtered, and concentrated. Purification by flash chromatography on SiO₂ (35% EtOAc/CH₂Cl₂) afforded 5-methoxy-3-(pyridin-2-ylmethyl)-1H-indole (54.7 mg, 0.230 mmol, 24%) as a tan-brown solid: (400 MHz, CDCl₃) δ 8.56 (d, J=4.2 Hz, 1H), 8.00 (bs, 1H), 7.54 (td, J=1.8, 7.7 Hz, 1H), 7.25 (d, J=9.8 Hz, 1H), 7.16 (d, J=7.8 Hz, 1H), 7.10 (dd, J=5.1, 7.2 Hz, 1H), 7.06 (d, J=2.2 Hz, 1H), 6.95 (d, J=2.4 Hz, 1H), 6.84 (dd, J=2.4, 8.8 Hz, 1H), 4.28 (s, 2H), 3.79 (s, 3H).

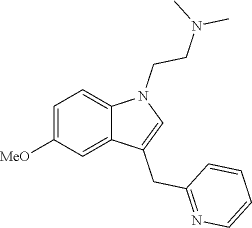 [SMILES: COc1ccc2c(c1)c(Cc1ccccn1)cn2CCN(C)C]

2-(5-methoxy-3-(pyridin-2-ylmethyl)-1H-indol-1-yl)-N,N-dimethylethan-1-amine: To a solution of 5-methoxy-3-(pyridin-2-ylmethyl)-1H-indole (50.0 mg, 0.210 mmol) in DMSO (0.53 mL) was added 2-chloro-M,N-dimethylamine hydrochloride (33.6 mg, 0.231 mmol), potassium iodide (38.7 mg, 0.231 equiv), and potassium hydroxide (59.4 mg, 1.05 mmol). The reaction was stirred at room temperature for 24 hours before being diluted with aqueous NaOH (1 M, 10 mL). The aqueous phase was extracted with CH₂Cl₂ (3×15 mL), and the combined organic extracts were dried (MgSO₄), filtered, and concentrated. Purification by flash column chromatography on SiO₂ (10% MeOH/EtOAc w/1% Et₃N) afforded 2-(5-methoxy-3-(pyridin-2-ylmethyl)-1H-indol-1-yl)-N,N-dimethylethan-1-amine (29.5 mg, 0.0953 mmol, 45%) as a brown oil: IR (ATR) νₘₐₓ 3415, 2940, 2824, 2769, 1621, 1591, 1568, 1486, 1452, 1434, 1221, 1153, 1100, 1038, 994, 895, 835, 768, 788, 751, 692 cm⁻¹; ¹H NMR (300 MHz, CDCl₃) δ 8.54 (d, J=4.1 Hz, 1H), 7.52 (td, J=1.8, 7.7 Hz, 1H), 7.21 (d, J=8.8 Hz, 1H), 7.14 (d, J=7.8 Hz, 1H), 7.08 (dd, J=5, 7.23 Hz, 1H), 7.00 (s, 1H), 6.93 (d, J=2.3 Hz, 1H), 6.85 (dd, J=2.4, 8.8 Hz 1H), 4.25-4.21 (m, 4H), 3.77 (s, 3H), 2.77 (t, J=7.2 Hz, 2H), 2.34 (s, 6H); ¹³C NMR (75 MHz, CDCl₃) δ 161.3, 153.9, 149.1, 136.5, 131.7, 128.4, 127.1, 122.8, 121.1, 112.5, 112.0, 110.0, 101.4, 58.7, 55.9, 45.4, 44.3, 34.6; HRMS (ESI+) m/z calculated for C₁₉H₂₄ON₃, [M+H]⁺ 310.19139, found 310.19086.

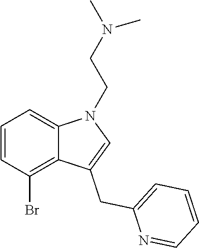 [SMILES: CN(C)CCn1cc(Cc2ccccn2)c2c(Br)cccc21]

2-(4-Bromo-3-(pyridin-2-ylmethyl)-1H-indol-1-yl)-N,N-dimethylethan-1-amine: To a solution of 4-bromo-3-(pyridin-2-ylmethyl)-1H-indole (0.200 g, 0.697 mmol) in DMSO (1.7 mL) were added 2-dimethylaminoethyl chloride hydrochloride (0.111 g, 0.766 mmol), KI (0.128 g, 0.766 mmol), and KOH (0.230 g, 3.48 mmol). The reaction was stirred at rt for 24 h before being diluted with EtOAc (20 mL). The organic later was washed with ice-cold brine (3×10 mL), dried (MgSO₄), filtered, and concentrated. Purification by chromatography on SiO₂ (17:3 EtOAc: MeOH with 1% Et₃N) afforded the product as a tan oil (0.0730 g, 0.204 mmol, 59%): IR (ATR) νₘₐₓ 3360, 3061, 2972, 2941, 2821, 2769, 1606, 1591, 1568, 1548, 1473, 1434, 1397, 1357, 1329, 1311, 1266, 1156, 1095, 1049, 1037, 1021, 994, 946, 832 cm⁻¹; ¹H NMR (500 MHz, CDCl₃) δ 8.57 (d, J=4.5 Hz, 1H), 7.56 (td, J=7.5, 1.5 Hz, 1H), 7.34 (d, J=8.5 Hz, 1H), 7.26-7.24 (m with overlap of residual CHCl₃, 1H), 7.13-7.11 (m, 2H), 7.05 (t, J=8.0 Hz, 1H), 6.97 (s, 1H), 4.86 (s, 2H), 4.46-4.28 (m, 2H), 3.02-3.17 (m, 2H), 2.43 (s, 6H); ¹³C NMR (125 MHz, CDCl₃) δ 162.0, 149.2, 137.8, 136.5, 128.9, 126.2, 123.9, 123.1, 122.7, 121.1, 114.8, 113.6, 108.8, 58.7, 45.6, 44.6, 35.0; HRMS (ESI+) m/z for C₁₈H₂₁N₃Br [M+H]+: calcd 358.09134; found 358.09146.

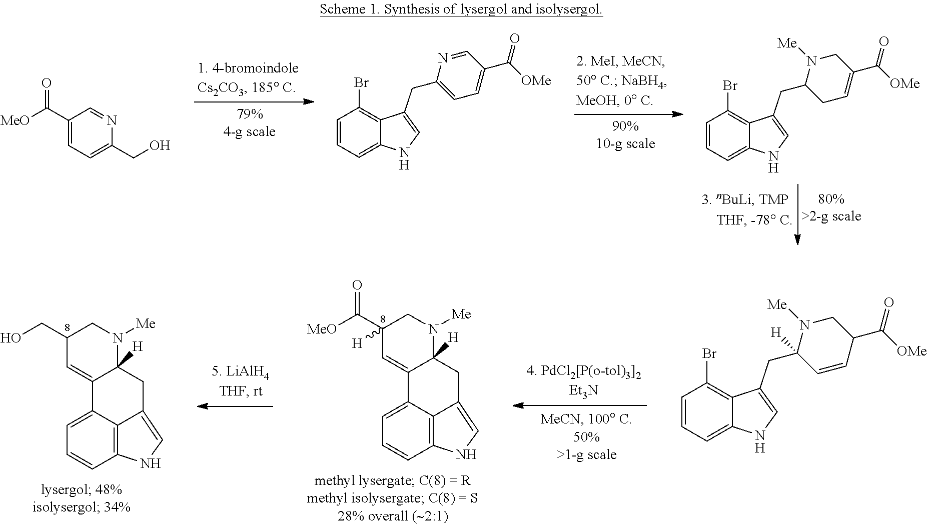 [SMILES: COC(=O)c1ccc(CO)nc1 ; COC(=O)c1ccc(Cc2c[nH]c3cccc(Br)c23)nc1 ; COC(=O)C1=CCC(Cc2c[nH]c3cccc(Br)c23)N(C)C1 ; CN1CC(CO)C=C2c3cccc4[nH]cc(c34)C[C@H]21 ; COC(=O)C1C=C2c3cccc4[nH]cc(c34)C[C@H]2N(C)C1 ; COC(=O)C1C=C[C@@H](Cc2c[nH]c3cccc(Br)c23)N(C)C1]

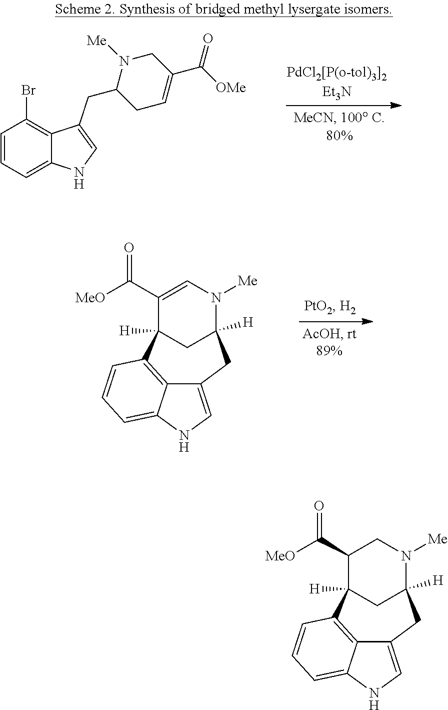 [SMILES: COC(=O)C1=CCC(Cc2c[nH]c3cccc(Br)c23)N(C)C1 ; COC(=O)C1=CN(C)[C@H]2Cc3c[nH]c4cccc(c34)[C@@H]1C2 ; COC(=O)[C@H]1CN(C)[C@H]2Cc3c[nH]c4cccc(c34)[C@@H]1C2]

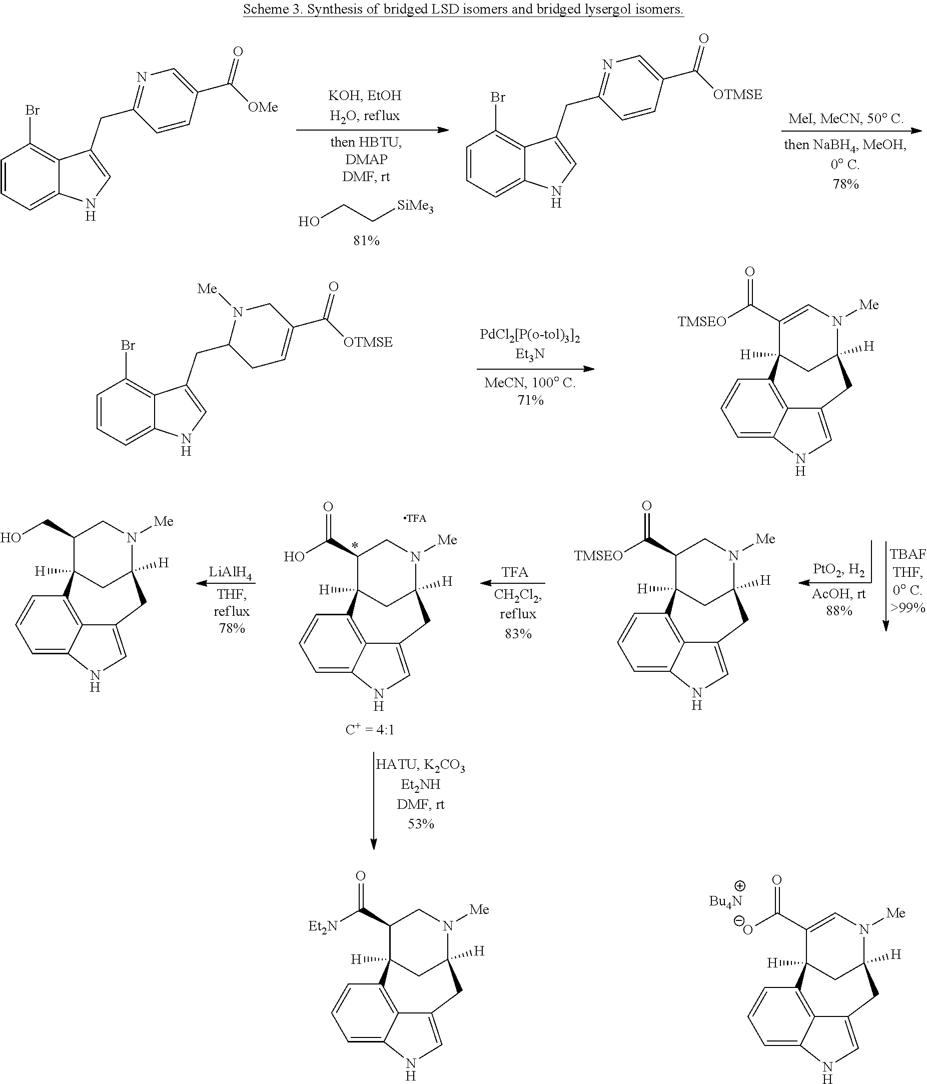

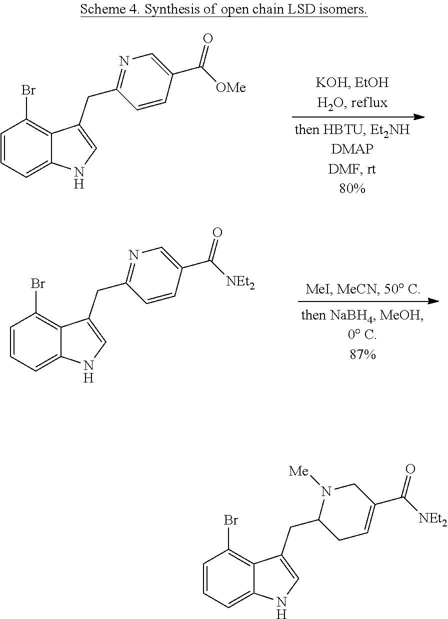

### Compound Preparation and Characterization

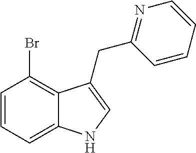 [SMILES: Brc1cccc2[nH]cc(Cc3ccccn3)c12]

4-Bromo-3-(pyridin-2-ylmethyl)-1H-indole. A mixture of 4-bromoindole (0.100 mL, 0.781 mmol), Cs₂CO₃ (0.280 g, 2.34 mmol) and 2-pyridinmethanol (0.231 mL, 2.34 mmol) was diluted with PhMe (0.39 mL, 2.0 M) in a brand new one-dram vial. The vial was sealed, and the mixture and heated at 110° C. for 24 h. The mixture was cooled to rt and diluted with CH₂Cl₂ (20 mL) and H₂O (10 mL). The aq. layer was extracted with 9:1 CH₂Cl₂:MeOH (2×10 mL), and the combined organic layers were washed with brine (30 mL), dried (MgSO₄), filtered, and concentrated. Purification by chromatography on SiO₂ (7:13 EtOAc:CH₂Cl₂) afforded the product as an off-white solid (0.201 g, 0.700 mmol, 90%): mp 185-186° C.; IR (ATR) νₘₐₓ 3112, 3021, 2917, 1725, 1596, 1559, 1543, 1473, 1434, 1424, 1409, 1331, 1249, 1174, 1042, 1002, 912, 843, 7043, 717 cm⁻¹; ¹H NMR (500 MHz, DMSO-d₆) δ 11.30 (brs, 1H), 8.48 (ddd, J=4.8, 1.7, 0.8 Hz, 1H), 7.63 (td, J=7.7, 1.9 Hz, 1H), 7.40 (dd, J=8.1, 0.8 Hz, 1H), 7.21 (d, J=2.6 Hz, 1H), 7.18-7.15 (m, 1H), 7.13 (dd, J=7.6, 0.8 Hz, 1H), 7.02 (d, J=7.8 Hz, 1H), 6.94 (t, J=7.9 Hz), 4.34 (s, 2H); ¹³C NMR (125 MHz, DMSO-d₆) δ 161.7, 148.7, 137.8, 136.3, 126.5, 124.8, 122.7, 122.3, 122.1, 120.9, 112.9, 112.1, 111.3, 34.3; HRMS (ESI+) m/z calcd for C₁₄H₁₂N₂Br [M+H]⁺ 287.0178, found 287.0175.

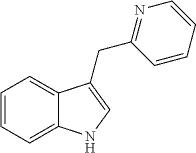 [SMILES: c1ccc(Cc2c[nH]c3ccccc23)nc1]

3-(Pyridin-2-ylmethyl)-1H-indole. ¹H NMR (300 MHz, CDCl₃) δ 8.56 (dd, J=4.8, 0.9 Hz, 1H), 8.12 (brs, 1H), 7.57-7.51 (m, 2H), 7.36 (d, J=8.1 Hz, 1H), 7.21-7.16 (m, 2H), 7.13-7.05 (m, 3H), 4.32 (s, 2H). Spectral data were consistent with literature properties.

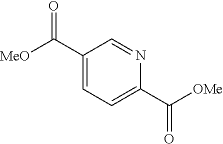 [SMILES: COC(=O)c1ccc(C(=O)OC)nc1]

Dimethyl pyridine-2,5-dicarboxylate. 2,5-dipyridinedicarboxylic acid (20.0 g, 117 mmol) was diluted with CH₂Cl₂ (300 mL), and SOCl₂ (25.6 mL, 352 mmol) was added. The solution was cooled to 0° C., and distilled DMF (0.910 mL, 11.7 mmol) was added. The solution was warmed to rt and heated at reflux (60° C. ext. temp) for 4 h, at which point the reaction mixture became homogenous. The reaction was cooled to 0° C., and MeOH (150 mL) was slowly added (dropwise) via addition funnel. After 30 min of stirring, the solvent was removed in vacuo, and the residue was partitioned between CH₂Cl₂ (300 mL) and sat. aq. NaHCO₃ (300 mL). The aq. layer was extracted with CH₂Cl₂ (2×100 mL), and the combined organic layers were washed with sat. aq. NaCl (200 mL), dried (MgSO₄), filtered, and concentrated to afford the product as a crystalline white solid (21.5 g, 110 mmol, 94%): ¹H NMR (400 MHz, CDCl₃) δ 9.30 (dd, J=2.0, 0.8 Hz, 1H), 8.45 (dd, J=8.4, 2.4=Hz, 1H), 8.21 (dd, J=8.0, 0.8 Hz, 1H), 4.04 (s, 3H), 3.99 (s, 3H). Spectral data were consistent with literature properties.

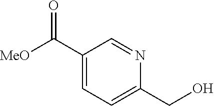 [SMILES: COC(=O)c1ccc(CO)nc1]

Methyl 6-(hydroxymethyl) nicotinate. Dimethyl pyridine-2,5-dicarboxylate (11.44 g, 58.46 mmol) was dissolved in MeOH (200 mL) and CH₂Cl₂ (200 mL). CaCl₂ (9.76 g, 87.9 mmol) was added. Finely ground NaBH₄ (6.65 g, 87.9 mmol) was added in 16 equal portions (0.416 g) over 8 h at rt. The resulting mixture was stirred at rt for 16 h (monitored by LC-MS). If needed, additional portions were added every hour until completion. The mixture was diluted with sat. aq. NH₄Cl (200 mL) and H₂O (200 mL) and stirred for 30 min. The layers were separated, and the aq. phase was extracted with CH₂Cl₂ (3×100 mL). The combined organic layers were dried (MgSO₄), filtered, and concentrated to afford the product as an off-white solid (8.69 g, 52 mmol, 89%): ¹H NMR (300 MHz, CDCl₃) δ 9.18 (d, J=1.5 Hz, 1H), 8.36 (d, J=8.1, 1H), 7.42 (d, J=8.1 Hz, 1H), 4.87 (s, 2H), 3.97 (s, 3H). Spectral data were consistent with literature properties.

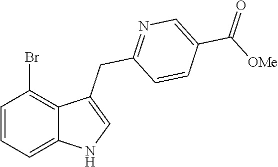 [SMILES: COC(=O)c1ccc(Cc2c[nH]c3cccc(Br)c23)nc1]

Methyl 6-((4-bromo-1H-indol-3-yl) methyl) nicotinate. Two 20 mL microwave vials were each charged with methyl 6-(hydroxymethyl) nicotinate (2.00 g, 12.0 mmol), 4-bromoindole (4.58 mL, 35.9 mmol), and Cs₂CO₃ (4.29 g, 13.2 mmol). The vials were sealed and stirred (egg-shaped stir bar) at 185±5° C. (aluminum block). The vessels were slowly vented every 10 min (three times) to release pressure buildup. After 24 h, the warm reactions were combined and diluted with MeOH (180 mL), and H₂SO₄ (10.2 mL, 191.4 mmol) was slowly added with vigorous stirring. The reaction mixture was stirred at reflux for 8 h. The reaction was diluted with CH₂Cl₂ (400 mL), and sat. aq. NaHCO₃ (250 mL) was slowly added with stirring. The aq. phase was extracted with 9:1 CH₂Cl₂:MeOH (4×100 mL), and the combined organic layers were dried (MgSO₄), filtered, and concentrated. Purification by flash column chromatography (2:3 EtOAc:Hexanes) afforded the product as a tan solid (6.50 g, 18.8 mmol, 79%): mp 171-173° C.; IR (ATR) νₘₐₓ 3139, 3031, 2950, 2922, 2882, 1726, 1597, 1561, 1483, 1427, 1381, 1284, 1199, 1175, 1115, 1329, 963, 949, 914, 846, 805, 760, 752 cm⁻¹; ¹H NMR (500 MHz, DMSO-d₆) δ 11.37 (brs, 1H), 9.01 (d, J=1.5 Hz, 1H), 8.15 (dd, J=8.0, 2.0 Hz, 1H), 7.41 (d, J=8.0 Hz, 1H), 7.32 (d, J=2.0 Hz, 1H), 7.13 (dd, J=7.0, 1.5 Hz, 2H), 6.98 (t, J=8.0 Hz, 1H), 4.52 (s, 2H), 3.86 (s, 3H); ¹³C NMR (125 MHz, DMSO-d₆) δ 166.9, 165.3, 149.5, 137.9, 137.1, 127.0, 124.8, 123.0, 122.8, 122.3, 122.2, 112.8, 111.4, 111.0, 52.2, 34.5; HRMS (ESI+) m/z calcd for C₁₆H₁₄O₂N₂Br [M+H]⁺ 345.0233, found 345.0233.

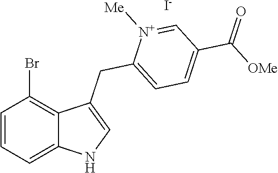 [SMILES: [I-] ; COC(=O)c1ccc(Cc2c[nH]c3cccc(Br)c23)[n+](C)c1]

2-((4-Bromo-1H-indol-3-yl) methyl)-5-(methoxycarbonyl)-1-methylpyridin-1-ium iodide. A solution of methyl 6-((4-bromo-1H-indol-3-yl) methyl) nicotinate (10.0 g, 29.0 mmol) and iodomethane (9.01 mL, 145 mmol) in MeCN (145 mL) was stirred at 50° C. in a sealed tube for 24 h. Upon completion, the solution was concentrated to afford the product as a yellow solid (14.1 g, 28.9 mmol, quant.). For characterization, an analytical sample was triturated with methanol: mp 205-207° C.; IR (ATR) νₘₐₓ 3165, 1727, 1636, 1560, 1430, 1294, 1262, 1171, 1147, 1044, 975, 914, 749, 739 cm⁻¹; ¹H NMR (300 MHz, DMSO-d₆) δ 11.65 (d, J=1.8 Hz, 1H), 9.63 (d, J=1.80 Hz, 1H), 8.77 (dd, J=7.5, 1.8 Hz, 1H), 7.53 (d, J=8.4 Hz, 1H), 7.51 (d, J=8.4 Hz, 1H), 7.48 (s, 1H), 4.80 (s, 2H), 4.50 (s, 3H), 3.97 (s, 3H); ¹³C NMR (125 MHz, DMSO-d₆) δ 162.7, 162.3, 147.9, 144.3, 138.0, 127.9, 126.9, 124.2, 123.1, 123.1, 112.4, 111.8, 106.2, 53.4, 45.9, 30.1; HRMS (ESI+) m/z calcd for C₁₇H₁₆O₂N₂Br, [M+H]⁺ 359.03897, found 359.03935.

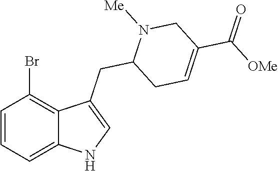 [SMILES: COC(=O)C1=CCC(Cc2c[nH]c3cccc(Br)c23)N(C)C1]

Methyl-((4-bromo-1H-indol-3-yl) methyl)-1-methyl-1,2,5,6-tetrahydropyridine-3-carboxylate. To a solution of crude 2-((4-bromo-1H-indol-3-yl) methyl)-5-(methoxycarbonyl)-1-methylpyridin-1-ium iodide (14.1 g, 28.9 mmol) in MeOH (145 mL) was added NaBH: (3.29 g, 86.8 mmol) in 6 equal portions over the course of 1 h at 0° C. The solution was stirred for an additional 1 h. The solution was diluted with H₂O (50 mL) and CH₂Cl₂ (300 mL). The aqueous layer was extracted with CH₂Cl₂ (3×50 mL), and the combined organic layers were washed with brine (50 mL), dried (MgSO₄), filtered, and concentrated. Purification by chromatography on SiO₂ (9:1 EtAOc/MeOH with 0.5% Et₃N) afforded the product as a dark yellow oil that became a tan foam upon drying under reduced pressure (9.47 g, 26.1 mmol, 91%): mp 57-59° C.; IR (ATR) νₘₐₓ 3346, 2924, 2796, 1706, 1656, 1614, 1559, 1434, 1335, 1264, 1190, 1140, 1096, 1023, 910, 800, 765, 722 cm⁻¹; ¹H NMR (500 MHz, CDCl₃) δ 8.25 (brs, 1H), 7.30 (dd, J=8.0 Hz, 1H), 7.27 (dd, J=8.0, 1.0 Hz, 1H), 7.06 (d, J=2.5 Hz, 1H), 7.00 (t, J=8.0 Hz, 1H), 6.98-6.96 (m, 1H), 3.75 (s, 3H), 3.56 (td, J=14.0, 4.0 Hz, 2H), 3.46 (dd, J=17.0, 4.0 Hz, 1H), 3.20-3.17 (m, 1H), 2.76 (dd, J=14.0, 10.0 Hz, 1H), 2.61 (s, 3H), 2.23-2.19 (m, 2H); ¹³C NMR (125 MHz, DMSO-d₆) δ 166.5, 137.8, 137.7, 128.0, 125.6, 124.8, 124.3, 123.0, 114.3, 113.8, 110.8, 57.9, 51.7, 51.4, 41.1, 28.8, 25.8; HRMS (ESI+) m/z calcd for C₁₇H₂₀O₂N₂Br [M+H]⁺ 363.0703, found 363.0699.

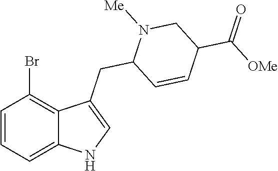 [SMILES: COC(=O)C1C=CC(Cc2c[nH]c3cccc(Br)c23)N(C)C1]

Methyl (6R)-6-((4-bromo-1H-indol-3-yl) methyl)-1-methyl-1,2,3,6-tetrahydropyridine-3-carboxylate. To a stirred solution of distilled 2,2,6,6-tetramethylpiperidine (7.34 mL, 42.9 mmol) in THF (40 mL) was added freshly titrated n-BuLi (15.6 mL, 35.8 mmol, 2.37 M in hexanes) at −78° C. The solution was warmed to 0° C. for 15 min then cooled to −78° C.

A solution of methyl 6-((4-bromo-1H-indol-3-yl) methyl)-1-methyl-1,2,5,6-tetrahydropyridine-3-carboxylate (2.60 g, 7.16 mmol) in dry THF (30 mL) was added drop-wise to the former solution via canula. Additional THF (10 mL) was used to rinse the flask and canula. After 2 h, 2,6-ditertbutyl phenol (10.4 g, 50.1 mmol) in THF (30.0 mL) was added slowly. The reaction mixture was warmed to 0° C. and stirred for 30 min. Sat. aq. NH₄Cl (30 mL) was added, followed by EtOAc (200 mL). The organic layer was separated and the aq. phase was extracted with EtOAc (2×30 mL). The combined organic phases were washed with brine (60 mL), dried (MgSO₄), filtered, and concentrated. Purification by chromatography on SiO₂ (19:1 EtOAc/MeOH with 0.5% Et₃N to elute product, then 19:1 EtOAc:MeOH with 0.5% Et₃N to elute starting material) afforded the product as a ˜1:1 mixture of diastereomers as a tan semi-solid that became a foam upon drying under reduced pressure (2.08 g, 5.73 mmol, 80%): ¹H NMR (500 MHz, CDCl₃) δ 8.18 (brs, 1H), 8.15 (brs, 1H), 7.31-7.27 (m, 4H), 7.15 (s, 2H), 7.01-6.97 (m, 2H), 5.86-5.84 (m, 2H), 5.76-5.70 (m, 2H), 3.74 (s, 3H), 3.70 (s, 3H) 3.70 (overlap with CH₃ singlet, 2H), 3.56 (dd, J=14.5, 5.0 Hz, 1H), 3.44 (m, 1H), 3.29-3.20 (m, 5H), 2.92 (dd, J=14.5, 9.0 Hz, 1H), 2.82-2.78 (m, 2H), 2.66 (t, J=10.5 Hz, 1H), 2.59 (s, 3H), 2.54 (s, 3H); ¹³C NMR (75 MHz, CDCl₃) δ 173.9, 173.3, 137.7, 137.7, 131.5, 131.2, 125.8, 125.8, 125.4, 125.2, 124.2, 124.1, 122.8, 122.7, 122.6, 122.1, 114.3, 113.5, 113.3, 110.7, 110.7, 62.1, 31.4, 54.3, 52.1, 52.1, 51.1, 43.2, 43.0, 41.9, 39.2, 29.4, 29.3; HRMS (ESI+) m/z calcd for C₁₇H₂₀O₂N₂Br [M+H]⁺ 363.0703, found 363.0701.

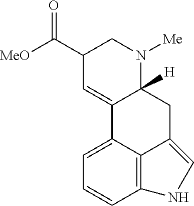 [SMILES: COC(=O)C1C=C2c3cccc4[nH]cc(c34)C[C@H]2N(C)C1]

Methyl (6aR)-7-methyl-4,6,6a,7,8,9-hexahydroindolo[4,3-fg]quinoline-9-carboxylate (methyl lysergate, methyl isolysergate). Methyl (6R)-6-((4-bromo-1H-indol-3-yl)methyl)-1-methyl-1,2,3,6-tetrahydropyridine-3-carboxylate (1.40 g, 3.85 mmol) and PdCl₂(P(o-tol)₃)₂ (0.319 g, 0.385 mmol) were suspended in degassed MeCN (80 mL), and Et₃N (1.64 mL, 11.6 mmol) was added. The reaction was stirred at 100° C. in a capped vial for 3 h. The solution was diluted with sat aq. NaHCO₃ (60 mL), H₂O (60 mL), and CH₂Cl₂ (200 mL). The aq. layer was extracted with CH₂Cl₂ (2×40 mL), and the combined organic layers were dried (MgSO₄), filtered, and concentrated. Purification by chromatography on SiO₂ (1:2 acetone/petroleum ether) afforded the product as a mixture of fluorescent (on TLC) diastereomers (0.564 g, 1.98 mmol, 50%): ¹H NMR (500 MHz, CDCl₃, 2:1 mixture of diastereomers) δ 7.95 (brs, 1.5 H), 7.24-7.17 (m, 4.5H), 6.91 (s, 1H), 6.90 (s, 0.5H), 6.60 (s, 1H), 6.56 (d, J=4.0 Hz, 0.5H), 3.78 (s, 3H), 3.72 (m with an apparent singlet, 2.5H), 3.52 (dd, J=14.5, 5.5 Hz, 1H), 3.43 (dd, J=14.5, 5.0 Hz, 0.5H), 3.38-3.35 (m with an apparent singlet at 3.35, 1H), 3.30-3.27 (m, 1.5H), 3.20-3.19 (m, 1.5H), 2.77-2.68 (m, 3H), 2.61 (s, 3H), 2.57 (s, 1.5H); ¹³C NMR (125 MHz, CDCl₃, 2:1 mixture of diastereomers) δ 173.6, 172.9, 138.8, 136.9, 136.2, 134.1, 128.9, 128.1, 126.5, 126.3, 123.5, 123.4, 118.4, 118.4, 118.2, 117.8, 112.7, 112.3, 111.5, 111.0, 109.8, 109.8, 63.0, 62.9, 56.1, 55.5, 53.2, 52.2, 43.8, 43.7, 42.2, 41.1, 27.2, 27.2; HRMS (ESI+) m/z calcd for C₁₇H₁₉O₂N₂ [M+H]⁺ 283.1441, found 283.1439.

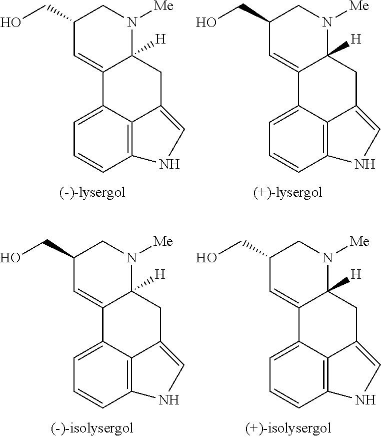 [SMILES: CN1C[C@@H](CO)C=C2c3cccc4[nH]cc(c34)C[C@@H]21 ; CN1C[C@H](CO)C=C2c3cccc4[nH]cc(c34)C[C@H]21 ; CN1C[C@H](CO)C=C2c3cccc4[nH]cc(c34)C[C@@H]21 ; CN1C[C@@H](CO)C=C2c3cccc4[nH]cc(c34)C[C@H]21]

Lysergol (1) and Isolysergol (2). A solution of methyl lysergate and methyl isolysergate (0.400 g, 1.42 mmol) in THF (16 mL) was cooled to 0° C., and LiAlH₄ in THF (1.30 mL, 3.12 mmol, 2.4 M) was added dropwise. The solution was stirred for an additional 30 min, followed by dropwise addition of EtOAc (10 mL) at 0° C. H₂O (10 mL) was slowly added, and the mixture was vigorously stirred for 10 min. The organic layer was transferred, dried (MgSO₄), filtered, and concentrated. Purification by chromatography on SiO₂ (9:1 CH₂Cl₂:MeOH) afforded isolysergol (0.124 g, 0.488 mmol, 34%) and lysergol (0.173 g, 0.681 mmol, 48%). Chiral resolution of lysergol and isolysergol was performed on a semi-prep OD-H column to provide (−)-lysergol [α]_{D}²⁵=−65.5 (c=0.32, methanol), (+)-lysergol [α]_{D}²⁵=+66.8 (c=0.32, methanol), (−)-isolysergol [α]_{D}²⁵=−130.5 (c=0.32, methanol), and (+)-isolysergol [α]_{D}²⁵=+142.1 (c=0.43, methanol).
Lysergol: IR (ATR) νₘₐₓ 3240, 3062, 2950, 2875, 2812, 2784, 1607, 1551, 1447, 1412, 1373, 1341, 1291, 1241, 1219, 1122, 1053, 1033, 948, 923, 895, 855 cm⁻¹; ¹H NMR (400 MHz, 8:2 CDCl₃:MeOD) δ 7.07 (dd, J=6.8, 2.0 Hz, 1H), 7.02-6.98 (m with an apparent singlet at 7.01, 2H), 6.78 (s, 1H), 6.25 (s, 1H), 3.53 (dd, J=10.8, 6.0 Hz, 1H), 3.43-3.63 (m, 2H), 3.20 (d, J=9.2 Hz, 1H), 2.99 (dd, J=10.8, 5.2 Hz, 1H), 2.73 (brs, 1H), 2.62-2.53 (m, 1H), 2.44 (s, 3H), 2.20-2.15 (m, 1H); ¹³C NMR (125 MHz, 8:2 CDCl₃:MeOD) δ 134.4, 134.0, 127.2, 125.9, 122.9, 121.1, 118.8, 111.8, 109.8, 109.0, 64.4, 63.2, 56.5, 43.0, 37.9, 26.2; HRMS (ESI+) m/z calcd for C₁₆H₁₉ON₂ [M+H]⁺ 255.14919, found 255.14967.
Isolysergol: IR (ATR) νₘₐₓ 3235, 2921, 2851, 1604, 1447, 1373, 1340, 1298, 1212, 1163, 1143, 1098, 1077, 1029, 985, 951, 914, 857, 837 cm⁻¹; ¹H NMR (400 MHz, 8:2 CDCl₃:MeOD) δ 7.19-7.15 (m, 1H), 7.11-7.01 (m, 2H), 6.87 (d, J=1.2 Hz, 1H), 6.41 (d, J=5.6 Hz, 1H), 3.90 (dd, J=10.4, 3.6 Hz, 1H), 3.76 (dd, J=10.4, 3.6 Hz, 1H), 3.51 (dd, J=14.4, 5.6 Hz, 1H), 3.25 (m, 1H), 3.10 (d, J=11.2 Hz, 1H), 2.87 (dd, J=11.6, 3.6 Hz, 1H), 2.70 (t, J=12.8 Hz, 1H), 2.59 (s, 3H), 2.49 (m, 1H); ¹³C NMR (125 MHz, 8:2 CDCl₃:MeOD) δ 136.3, 134.0, 127.7, 126.0, 122.9, 120.7, 118.7, 111.7, 109.8, 109.3, 65.4, 63.1, 56.5, 43.2, 36.3, 27.1; HRMS (ESI+) m/z caled for C₁₆H₁₉ON₂ [M+H]⁺ 255.14919, found 255.14955.

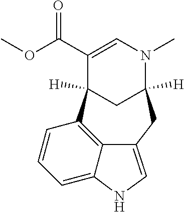 [SMILES: COC(=O)C1=CN(C)[C@H]2Cc3c[nH]c4cccc(c34)[C@@H]1C2]

Methyl (6S,10R)-9-methyl-6,9,10,11-tetrahydro-2H-6,10-methanoazonino[4,5,6-cd]indole-7-carboxylate. To a solution of methyl 6-((4-bromo-1H-indol-3-yl) methyl)-1-methyl-1,2,5,6-tetrahydropyridine-3-carboxylate (0.050 g, 0.14 mmol) and PdCl₂(P(o-tol)₃)₂ (0.011 g, 0.014 mmol) in MeCN (3.0 mL) was added Et₃N (0.0388 mL, 0.275 mmol). The solution was stirred at 100° C. in a sealed tube for 3 h. The solution was diluted with CH₂Cl₂ (15 mL) and washed with NaHCO₃ (10 mL). The aq. phase was extracted with CH₂Cl₂ (2×5 mL), dried (MgSO₄), filtered, and concentrated. Purification by chromatography on SiO₂ (3:2 EtOAc: Hexanes with 1% Et₃N) afforded the product as a tan solid (0.031 g, 0.11 mmol, 80%): mp 210-212° C.; IR (ATR) νₘₐₓ 3254, 3046, 2914, 1655, 1606, 1499, 1438, 1427, 1338, 1326, 1295, 1249, 1233, 1214, 1191, 1168, 1119, 1097, 1067, 1016, 951, 918, 852, 789, 766, 744, 725 cm⁻¹; ¹H NMR (500 MHz, CDCl₃) δ 8.01 (brs, 1H), 7.37 (d, J=7.0 Hz, 1H), 7.22 (s, 1H), 7.16 (dd, J=8.0, 1.0 Hz, 1H), 7.09 (dd, J=7.0, 0.5 Hz, 1H), 6.98 (t, J=1.5 Hz, 1H), 4.38 (d, J=5.0 Hz, 1H), 3.76-3.73 (m, 1H), 3.60 (s, 3H), 3.54 (dd, J=19, 2.5 Hz, 1H), 3.03 (s, 3H), 2.74 (dq, J=16.5, 1.5 Hz, 1H), 2.44 (d, J=13.5 Hz, 1H), 2.41-2.35 (m, 1H); ¹³C NMR (125 MHz, CDCl₃) δ 168.2, 145.0, 138.2, 137.5, 124.5, 122.1, 122.0, 120.1, 111.5, 108.8, 98.3, 54.2, 50.4, 41.6, 35.3, 31.3, 31.1; HRMS (ESI+) m/z for calcd for C₁₇H₁₉O₂N₂ [M+H]⁺ 283.1441, found 283.1438.

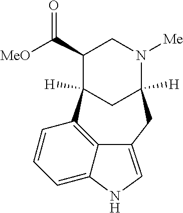 [SMILES: COC(=O)[C@H]1CN(C)[C@H]2Cc3c[nH]c4cccc(c34)[C@@H]1C2]

Methyl (6R,10R)-9-methyl-6,7,8,9,10,11-hexahydro-2H-6,10-methanoazonino[4,5,6-cd]indole-7-carboxylate. PtO₂ hydrate (0.080 g, 0.28 mmol) and methyl (6S, 10R)-9-methyl-6,9,10,11-tetrahydro-2H-6,10-methanoazonino[4,5,6-cd]indole-7-carboxylate (0.800 g, 2.83 mmol) were suspended in AcOH (14 mL). The suspension was sparged with N₂ for 5 min, followed by H₂ for 5 min, and the solution was stirred under an atmosphere of H₂ (1 atm, balloon) for 16 h. Upon completion, the mixture was filtered over Celite (MeOH) and concentrated. The AcOH was co-evaporated with PhMe (2×50 mL). The residue was diluted with CH₂Cl₂ (50 mL), and sat. aq. NaHCO₃ (100 mL) was slowly added. The aq. phase was extracted with 9:1 CH₂Cl₂/MeOH (2×25 mL), and the combined organic layers were dried (MgSO₄), filtered, and concentrated. The residue was purified by chromatography on C18 (10-100% MeCN/H₂O over 20 CV). The volatiles were removed in vacuo, and the solution was diluted with sat. aq. NaCl (1 volume) and extracted with 9:1 CH₂Cl₂:MeOH (3×25 mL). The combined organic layers were dried (MgSO₄), filtered, and concentrated to afford the product as an off-white solid (0.719 g, 2.53 mmol, 89%): mp 191-193° C.; IR (ATR) νₘₐₓ 3017, 2926, 2868, 2743, 1727, 1707, 1617, 1579, 1431, 1387, 1374, 1431, 1387, 1374, 1333, 1315, 1276, 1258, 1234, 1196, 1175, 1156, 1133, 1037, 1001, 969, 886, 863, 832, 749, 727, 665 cm⁻¹; ¹H NMR (500 MHz, CDCl₃) δ 7.98 (brs, 1H), 7.21 (d, J=8.0 Hz, 1H), 7.04 (t, J=7.5 Hz, 1H), 7.02 (s, 1H), 3.88-3.87 (t, J=3.5 Hz, 1H), 3.65 (d, J=17.0 Hz, 1H), 3.51 (s, 3H), 3.40 (m, 1H), 3.18-3.16 (m, 1H), 2.66-2.61 (m, 3H), 2.56 (s, 3H), 2.52-2.49 (m, 1H), 2.39 (d, J=14.0 Hz, 1H); ¹³C NMR (125 MHz, CDCl₃) δ 173.4, 136.9, 134.4, 128.2, 121.6, 121.5, 119.8, 115.3, 109.8, 54.6, 51.2, 49.4, 47.5, 42.7, 41.0, 35.0, 27.4; HRMS (ESI+) m/z calcd for C₁₇H₂₁O₂N₂, [M+H]⁺ 285.15975, found 285.16028.

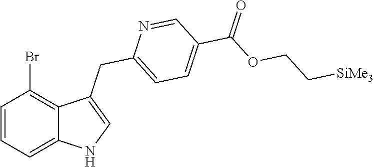 [SMILES: C[Si](C)(C)CCOC(=O)c1ccc(Cc2c[nH]c3cccc(Br)c23)nc1]

2-(Trimethylsilyl) ethyl 6-((4-bromo-1H-indol-3-yl) methyl) nicotinate. A solution of methyl 6-((4-bromo-1H-indol-3-yl) methyl) nicotinate (3.80 g, 11.0 mmol) and KOH (0.856 g, 13.2 mmol) in EtOH/H₂O (55 mL, 4:1) was heated to 70° C. for 16 h. The solution was concentrated, diluted with PhMe, concentrated a second time, and dried under high vacuum. HBTU (8.37 g, 22.0 mmol) and DMF (40 mL) were then added. The solution was stirred at rt for 1 h, and 4-DMAP (2.69 g, 22.0 mmol) and 2-(trimethylsilyl) ethanol (2.37 mL, 16.5 mmol) were added. The solution was stirred at rt for 12 h. The solution was diluted with EtOAc (300 mL) and sat. aq. NaHCO₃ (50 mL), and the layers were separated. The organic layer was washed with 1:1 water/brine (5×80 mL), dried (MgSO₄), filtered, and concentrated. Purification by chromatography on SiO₂ (7:13 EtOAc/Hexanes) afforded the product as a tan semi-solid (3.84 g, 8.90 mmol, 81%): mp 155-157° C.; IR (ATR) νₘₐₓ 3140, 3031, 2957, 2923, 2891, 1720, 1601, 1565, 1485,, 1464, 1451, 1380, 1336, 1279, 1246, 1200, 1120, 1060, 1029, 928, 919, 836 cm⁻¹; ¹H NMR (500 MHz, CDCl₃) δ 9.17 (d, J=2.0 Hz, 1H), 8.49 (brs, 1H), 8.15 (dd, J=8.5, 2.5 Hz, 1H), 7.30 (d, J=8.0 Hz, 1H), 7.24 (d, J=7.5 Hz, 1H), 7.18 (d, J=8.5 Hz, 1H), 7.04 (d, J=2.0 Hz, 1H), 7.00 (t, J=8.5 Hz, 1H), 4.65 (s, 2H), 4.43 (dd, J=10.0, 3.5 Hz, 2H), 1.14-1.11 (m, 2H), 0.08 (s, 9H); ¹³C NMR (125 MHz, CDCl₃) δ 166.5, 165.8, 150.3, 137.9, 137.7, 125.6, 125.4, 124.3, 124.2, 123.3, 122.8, 114.3, 113.4, 110.8, 63.7, 35.2, 17.6, −1.3; HRMS (ESI+) m/z calcd for C₂₀H₂₄O₂N₂BrSi [M+H]⁺ 431.07849; found 431.07798.

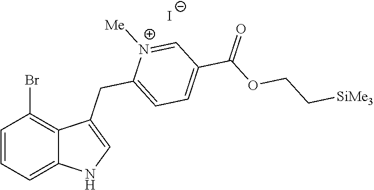 [SMILES: [I-] ; C[n+]1cc(C(=O)OCC[Si](C)(C)C)ccc1Cc1c[nH]c2cccc(Br)c12]

2-((4-Bromo-1H-indol-3-yl) methyl)-1-methyl-5-((2-(trimethylsilyl)ethoxy)carbonyl)pyridin-1-ium iodide. A solution of 2-(trimethylsilyl)ethyl 6-((4-bromo-1H-indol-3-yl) methyl) nicotinate (3.00 g, 6.95 mmol) and iodomethane (2.16 mL, 34.8 mmol) in MeCN (70 mL) was stirred at 50° C. for 24 h in a closed tube. After 24 h, the solution was concentrated to afford the product as a yellow solid and was carried forward without further purification. An analytical sample was recrystallized from MeCN: mp 121-123° C.; IR (ATR) νₘₐₓ 3188, 2952, 1725, 1636, 1518, 1422, 1334, 1292, 1249, 1173, 1137, 1068, 1043, 914, 857, 836 cm⁻¹; ¹H NMR (500 MHz, DMSO-d₆) δ 11.64 (d, J=2.0 Hz, 1H), 9.59 (d, J=1.5 Hz, 1H), 8.75 (dd, J=8.5, 2.0 Hz, 1H), 7.55 (1H, J=8.5 Hz, 1H), 7.49 (d, J=8.5, 1H), 7.48 (d, J=2.5 Hz, 1H), 7.18 (d, J=7.5 Hz, 1H), 7.06 (t, J=8.0 Hz, 1H), 4.80 (s, 2H), 4.50-4.46 (m with an apparent singlet at 4.50, 5H), 1.15-1.12 (m, 2H), 0.07 (s, 9H); ¹³C NMR (125 MHz, DMSO-d₆) δ 162.6, 161.8, 147.9, 144.2, 138.1, 128.0, 128.0, 127.2, 124.2, 123.1, 123.1, 112.4, 111.8, 106.2, 64.7, 46.0, 30.2, 17.0, −1.5; HRMS (ESI+) m/z for calcd C₂₁H₂₆O₂N₂BrSi [M+H]⁺ 445.09414, found 445.09314.

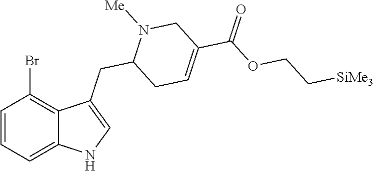 [SMILES: CN1CC(C(=O)OCC[Si](C)(C)C)=CCC1Cc1c[nH]c2cccc(Br)c12]

2-(Trimethylsilyl)ethyl 6-((4-bromo-1H-indol-3-yl)methyl)-1-methyl-1,2,5,6-tetrahydropyridine-3-carboxylate. To a solution of crude 2-((4-bromo-1H-indol-3-yl)methyl)-1-methyl-5-((2-(trimethylsilyl)ethoxy)carbonyl)pyridin-1-ium iodide (3.98 g, 8.92 mmol) in MeOH (45 mL) was added NaBH₄ (0.789 g, 26.7 mmol) in three equal portions (263 mg) over the course of 1 h at 0° C. The solution was stirred for an additional 30 min. The solution was diluted with H₂O (40 mL) and CH₂Cl₂ (120 mL). The aqueous layer was extracted with CH₂Cl₂ (2×40 mL), and the combined organic layers were dried (MgSO₄), filtered, and concentrated. Purification by chromatography on SiO₂ (97:3 EtOAc/MeOH w/1% Et₃N) afforded the product as a tan oil that became solid under vacuum (2.43 g, 3.12 mmol, 78%): mp 48-50° C.; IR (ATR) νₘₐₓ 3341, 3135, 3024, 2952, 2897, 2800, 1702, 1657, 1615, 1560, 1422, 1335, 1249, 1191, 1158, 1141, 1094, 1065, 1043, 1018, 933, 912, 856, 835 cm⁻¹; ¹H NMR (500 MHz, CDCl₃) δ 8.25 (brs, 1H), 7.31-7.28 (m, 2H), 7.05 (d, J=2.5 Hz, 1H), 7.00 (t, J=8.0 Hz, 1H), 6.95-9.64 (m, 1H), 4.26-4.23 (m, 2H), 3.55 (td, J=14.5, 4.0 Hz, 2H), 3.34 (d, J=17.0 Hz, 1H), 3.17 (dd, J=9.5, 4.5 Hz, 1H), 2.76 (dd, J=14.0, 10.5 Hz, 1H), 2.60 (s, 3H), 2.20-2.18 (m, 2H), 1.04-1.01 (m, 2H), 0.05 (s, 9H); ¹³C NMR (125 MHz, CDCl₃) δ 166.3, 137.8, 137.2, 128.5, 125.6, 124.8, 124.3, 123.0, 114.4, 113.9, 110.7, 62.8, 57.9, 51.5, 41.1, 28.9, 25.8, 17.5, −1.3; HRMS (ESI+) m/z calcd for C₂₁H₃₀O₂N₂BrSi [M+H]⁺ 449.12544; found 449.12482.

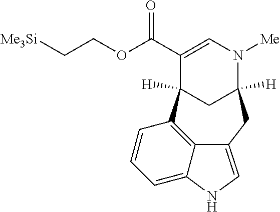 [SMILES: CN1C=C(C(=O)OCC[Si](C)(C)C)[C@H]2C[C@@H]1Cc1c[nH]c3cccc2c13]

2-(Trimethylsilyl)ethyl (6S,10R)-9-methyl-6,9,10,11-tetrahydro-2H-6,10-methanoazonino[4,5,6-cd]indole-7-carboxylate. To solution of 2-(trimethylsilyl)ethyl 6-((4-bromo-1H-indol-3-yl) methyl)-1-methyl-1,2,5,6-tetrahydropyridine-3-carboxylate (2.43 g, 5.41 mmol) and PdCl₂[P(o-tol)₃]₂ (0.224 g, 0.270 mmol) suspended in MeCN (110 mL) was added Et₃N (2.30 mL, 16.2 mmol). The solution was stirred at 100° C. for 3 h in a sealed tube. The solution was concentrated to ˜⅓ volume and diluted with CH₂Cl₂ (150 mL) and sat. aq. NaHCO₃ (50 mL). The aq. phase was extracted with CH₂Cl₂ (2×40 mL), dried (MgSO₄), filtered, and concentrated. Purification by chromatography on SiO₂ (2:3 EtOAc:Hexanes) afforded the product as a tan solid (1.53 g, 4.15 mmol, 77%): IR (ATR) νₘₐₓ 3400, 3299, 3001, 2949, 1656, 1610, 1414, 1337, 1320, 1249, 1214, 1161, 1120, 1096, 1049, 938, 853, 834 cm⁻¹; ¹H NMR (500 MHz, CDCl₃) δ 8.00 (brs, 1H), 7.39 (d, J=7.0 Hz, 1H), 7.21 (s, 1H), 7.16 (d, J=8.0 Hz, 1H), 7.09 (t, J=7.5 Hz, 1H), 6.97 (s, 1H), 4.38 (d, J=4.5 Hz, 1H), 4.21-4.15 (m, 1H), 4.06-4.00 (m, 1H), 3.74-3.73 (m, 1H), 3.54 (dd, J=16.5, 2.5 Hz, 1H), 3.03 (s, 3H), 2.74 (dd, J=15, 3.5 Hz, 1H), 2.45-2.35 (m, 2H), 1.00-0.95 (m, 2H), 0.01 (s, 9H); ¹³C NMR (125 MHz, CDCl₃) δ 168.0, 144.8, 138.3, 137.5, 124.6, 122.2, 121.9, 120.3, 111.6, 108.7, 98.8, 60.9, 54.3, 41.6, 35.3, 31.4, 31.2, 17.8, −1.3; HRMS (ESI+) m/z calcd for C₂₁H₂₉O₂N₂Si [M+H]⁺ 369.19928; found 369.19929.

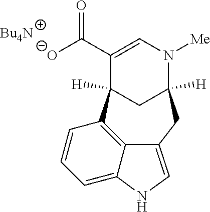 [SMILES: CN1C=C(C(=O)[O-])[C@H]2C[C@@H]1Cc1c[nH]c3cccc2c13]

Tetrabutylammonium (6S,10R)-9-methyl-6,9,10,11-tetrahydro-2H-6,10-methanoazonino[4,5,6-cd]indole-7-carboxylate. A solution of 2-(trimethylsilyl)ethyl (6S, 10R)-9-methyl-6,9,10,11-tetrahydro-2H-6,10-methanoazonino[4,5,6-cd]indole-7-carboxylate (0.960 g, 2.60 mmol) in THF (26 mL) was cooled to 0° C. TBAF (5.21 mL, 0.30 mmol, 1 M in THF) was added, and the solution was heated at reflux overnight. Upon completion, the solution was concentrated under a stream of N₂, and the residue was suspended in THF (5 mL) and cooled to 0° C. The solid was filtered and washed with THF (2×2 mL). The solid was collected to afford the product as a white powder (0.975 g, 1.91 mmol, 73%): mp 209-211° C.; IR (ATR) νₘₐₓ 3339, 2957, 2919, 2870, 1877, 1635, 1545, 1479, 1359, 1329, 1316, 1223, 1195, 1156, 1136, 1089, 1063, 1038, 1008, 811, 797, 749 cm⁻¹; ¹H NMR (500 MHz, DMSO-d₆) δ 10.95 (brs, 1H), 7.25 (d, J=7.5 Hz, 1H), 6.99 (d, J=8.0 Hz, 1H), 6.95 (s, 1H), 6.76 (t, J=7.5 Hz, 1H), 6.48 (s, 1H), 4.32 (d, J=4.5 Hz, 1H), 3.49-3.48 (m, 1H), 3.41 (d, J=16.0 Hz, 1H), 3.16 (dd, J=8.5, 8.5 Hz, 8H), 2.78 (d, 3H), 2.54-2.50 (m, 1H), 2.15 (d, J=8.0 Hz, 1H), 2.09-2.04 (m, 1H), 1.56 (p, J=7.5 Hz, 8H), 1.30 (sex, J=7.5 Hz, 8H), 0.93 (t, J=7.5 Hz, 12H); ¹³C NMR (125 MHz, DMSO-d₆) δ 170.4, 139.3, 137.2, 137.1, 124.8, 121.9, 120.0, 119.0, 111.0, 109.6, 107.4, 57.5, 53.3, 40.6, 36.4, 31.6, 31.6, 23.0, 19.2, 13.5; HRMS (ESI+) m/z calcd for C₁₆H₁₇O₂N₂ [M+H]⁺ 269.12845, found 269.12831.

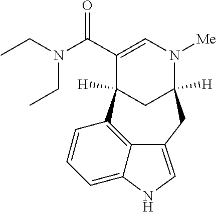 [SMILES: CCN(CC)C(=O)C1=CN(C)[C@H]2Cc3c[nH]c4cccc(c34)[C@@H]1C2]

(6S,10R)-N,N-Diethyl-9-methyl-6,9,10,11-tetrahydro-2H-6,10-methanoazonino[4,5,6-cd]indole-7-carboxamide. Tetrabutylammonium (6S,10R)-9-methyl-6,9,10,11-tetrahydro-2H-6,10-methanoazonino[4,5,6-cd]indole-7-carboxylate (0.300 g, 0.589 mmol) and HBTU (0.335 g, 0.883 mmol) were diluted with DMF (6 mL). The solution was stirred at rt for 1 h. Diethylamine (0.122 mL, 1.18 mmol) was added, and the solution was stirred at 80° C. in a sealed tube for 16 h. The solution was diluted with EtOAc (40 mL) and washed with brine (5×12 mL). The organic layer was dried (MgSO₄), filtered, and concentrated. Purification by chromatography on C18 (10-100% MeCN/H₂O) followed by chromatography on SiO₂ (1:1 Ac/Hex) afforded the product as a white foam (0.083 g, 0.257 mmol, 44%): mp 113-115° C.; IR (ATR) νₘₐₓ 3258, 2971, 2932, 1637, 1571, 1459, 1429, 1399, 1343, 1314, 1289, 1235, 1206, 1097, 910, 729 cm⁻¹; ¹H NMR (500 MHz, CDCl₃) δ 8.00 (brs, 1H), 7.11 (dd, J=8.0, 0.5 Hz), 7.00 (d, J=7.0 Hz, 1H), 6.98 (s, 1H), 6.92 (1H, J=7.0 Hz, 1H), 6.11 (s, 1H), 4.39 (t, J=3.0 Hz, 1H), 3.68-3.66 (m, 1H), 3.48 (dd, J=16.5, 3.5 Hz, 1H), 3.08-3.02 (m, 2H), 2.95-2.88 (m with an apparent singlet at 2.92, 5H), 2.77-2.73 (m, 1H), 2.43 (t, J=3.5 Hz, 2H), 0.83 (t, J=7.5 Hz, 6H); ¹³C NMR (125 MHz, CDCl₃) δ 173.8, 138.2, 137.6, 135.6, 124.5, 122.3, 122.0, 118.3, 112.2, 108.6, 103.7, 54.1, 41.6, 41.3, 37.9, 31.6, 30.6, 13.5; HRMS (ESI+) m/z calcd for C₂₀H₂₆ON₃ [M+H]⁺ 324.20704, found 324.20544.

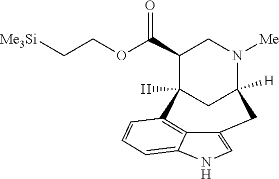

2-(Trimethylsilyl)ethyl (6R,7R,10R)-9-methyl-6,7,8,9,10,11-hexahydro-2H-6,10-methanoazonino[4,5,6-cd]indole-7-carboxylate. PtO₂ hydrate (0.0570 g, 0.201 mmol) and 2-(trimethylsilyl)ethyl (6S,10R)-9-methyl-6,9,10,11-tetrahydro-2H-6,10-methanoazonino[4,5,6-cd]indole-7-carboxylate (1.48 g, 4.02 mmol) were suspended in AcOH (12.0 mL). The mixture was sparged with N₂ for 5 min, followed by H₂ for 5 min, and the solution was stirred under an atmosphere of H₂ (1 atm, balloon) for 16 h. The reaction was diluted with CH₂Cl₂ (30 mL), and the solution was slowly added to sat. aq. NaHCO₃ (150 mL), and the aq. phase was extracted with CH₂Cl₂ (3×25 mL). The combined organic layers were dried (MgSO₄), filtered, and concentrated. The residue was filtered on a two-inch plug of SiO₂ (Acetone) to afford the product as an off-white solid (1.31 g, 3.52 mmol, 88%): mp 111-113° C.; IR (ATR) νₘₐₓ 3144, 3054, 2920, 2873, 1740, 1723, 1618, 1440, 1431, 1393, 1333, 1322, 1245, 1171, 1153, 1137, 1123, 1081, 1063, 1039, 995, 930, 835 cm⁻¹; ¹H NMR (500 MHz, CDCl₃) δ 7.99 (brs, 1H), 7.19 (d, J=8.0 Hz, 1H), 7.01 (t, J=8.0 Hz, 1H), 6.98 (s, 1H), 6.82 (d, J=7.0 Hz, 1H), 4.00-3.96 (m, 2H), 3.87 (t, J=4.0 HZ, 1H), 3.65 (d, J=17.0 Hz, 1H), 3.39 (brs, 1H), 3.12-3.09 (m, 1H), 2.65-2.58 (m, 3H), 2.55 (s, 3H), 2.47 (dd, J=12.0, 3.5 Hz, 1H), 2.39 (d, J=19.0 Hz, 1H), 0.86 (dd, 2H, J=9.0, 7.0 Hz, 2H), 0.00 (s, 9H); ¹³C NMR (75 MHz, CDCl₃) δ 173.2, 136.9, 134.5, 121.6, 121.4, 120.1, 115.4, 109.8, 62.3, 54.6, 49.5, 47.5, 42.7, 41.0, 35.1, 27.4, 17.4, −1.4; HRMS (ESI+) m/z calcd for C₂₁H₃₁O₂N₂Si [M+H]⁺ 371.21493, found 371.21461.

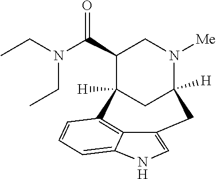

(6R,7R,10R)-N,N-Diethyl-9-methyl-6,7,8,9,10,11-hexahydro-2H-6,10-methanoazonino[4,5,6-cd]indole-7-carboxamide. TFA (1.2 mL) was added to a solution of 2-(trimethylsilyl)ethyl (6R,7R,10R)-9-methyl-6,7,8,9,10,11-hexahydro-2H-6,10-methanoazonino[4,5,6-cd]indole-7-carboxylate (0.580 g, 1.57 mmol) in CH₂Cl₂ (6 mL), and the reaction mixture was stirred at reflux for 24 h. The volatiles were removed under reduced pressure. The solid residue was suspended in CH₂Cl₂ (5 mL), stirred, and filtered to give (6R,7R,10R)-7-carboxy-9-methyl-6,7,8,9,10,11-hexahydro-2H-6,10-methanoazonino[4,5,6-cd]indol-9-ium trifluoroacetate as an off-white solid (0.675 g, 1.76 mmol, 99%) which was carried forward without further purification.

A solution of (6R,7R,10R)-7-carboxy-9-methyl-6,7,8,9,10,11-hexahydro-2H-6,10-methanoazonino[4,5,6-cd]indol-9-ium trifluoroacetate (0.100 g, 0.260 mmol), K₂CO₃ (0.0900 g, 0.650 mmol), and HATU (0.150 g, 0.390 mmol) in DMF (2.6 mL) was stirred at rt for 30 min, at which time Et₂NH (0.0404 mL, 0.390 mmol) was added. The solution was stirred for 16 h, diluted with EtOAc (30 mL), and washed with H₂O (5×6 mL). The organic layer was washed with brine (6 mL), dried (filtered), and concentrated. Purification by chromatography on C18 (0-100% MeOH/H₂O) afforded (6R,7R,10R)-N,N-diethyl-9-methyl-6,7,8,9,10,11-hexahydro-2H-6,10-methanoazonino[4,5,6-cd]indole-7-carboxamide as an off-white solid (0.0452 g, 0.138 mmol, 53%): mp 214-216° C.; IR (ATR) νₘₐₓ 3256, 2971, 2917, 1624, 1608, 1462, 1432, 1373, 1352, 1335, 1290, 1235, 1162, 1139, 1119, 1094, 1067, 1042, 916, 955, 871 cm⁻¹; ¹H NMR (500 MHz, CDCl₃) δ 8.77 (brs, 1H), 6.87-6.82 (m, 2H), 6.70 (m, 1H), 6.58 (dd, J=6.0, 1.5 Hz, 1H), 3.78 (sex, J=7.0 Hz, 1H), 3.70 (sex, J=7.0 Hz, 1H), 3.67-3.61 (m, 2H), 3.46-3.39 (m, 2H), 3.35-3.31 (m, 1H), 3.04 (t, J=12.5 Hz, 1H), 2.95 (sex, J=7.0 Hz, 1H), 2.66-2.60 (m with an apparent singlet at 2.66, 5H), 2.52 (dd, J=12.0, 3.0 Hz, 1H), 2.41 (d, J=14.0 Hz, 1H), 1.32 (t, J=7.0 Hz, 3H), 1.20 (t, J=7.0 Hz, 3H); ¹³C NMR (125 MHz, CDCl₃) δ 172.0, 137.0, 133.1, 127.7, 122.2, 120.3, 119.5, 113.2, 110.4, 55.1, 49.2, 46.8, 42.7, 41.7, 41.1, 39.8, 35.2, 27.2, 14.8, 13.1; HRMS (ESI+) m/z calcd for C₂₀H₂₈ON₃ [M+H]⁺ 326.22269, found 326.22254.

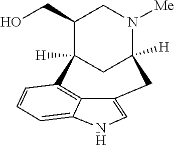

((6SR,10RS)-9-methyl-6,9,10,11-tetrahydro-2H-6,10-methanoazonino[4,5,6-cd]indol-7-yl) methanol. To a solution of (6SR,10RS)-7-carboxy-9-methyl-6,9,10,11-tetrahydro-2H-6,10-methanoazonino[4,5,6-cd]indol-9-ium trifluoroacetate (0.100 g, 0.260 mmol) in THF (3 mL) was added LAH in THF (0.542 mL, 1.30 mmol, 2.4 M). The solution was stirred at reflux overnight. Upon completion, the reaction was diluted with EtOAc (5 mL) and stirred for 15 min. 1 M NaOH (3 mL) was added and the reaction was stirred for 15 min. The organic layer was collected, dried (MgSO₄), filtered over Celite, and concentrated. Purification by chromatography on SiO₂ (7:3 CH₂Cl₂:MeOH) afforded the product as a white solid (0.0522 g, 0.202 mmol, 78%): mp >180° C. decomp; IR (ATR) νₘₐₓ 3316, 3145, 3058, 2992, 2923, 2909, 2870, 2746, 1617, 1575, 1474, 1461, 1440, 1429, 1377, 1343, 1331, 1315, 1272, 1241, 1156, 1134, 1081, 1067, 1024, 962 cm⁻¹; ¹H NMR (400 MHz, CD₃OD) δ 7.91 (s, 1H), 7.22 (dd, J=8.0, 1.0 Hz, 1H), 7.06 (d, J=1.2 Hz, 1H), 7.00 (apparent t, J=7.2 Hz, 1H), 6.82 (d, J=7.2 Hz, 1H), 3.69 (d, J=17.2 Hz, 1H), 3.50-3.45 (m, 2H), 3.31-3.28 (m overlap with residual CH₃OH, 1H), 2.77 (ddd, J=17.2, 4.0, 2.4 Hz, 1H), 2.64-2.56 (m with an apparent singlet at 2.60, 4H), 2.55 (d, J=12.0 Hz, 1H), 2.38 (d, J=14.0 Hz, 1H), 2.28 (m, 2H); ¹³C NMR (100 MHz, CD₃OD) δ 138.8, 134.5, 129.2, 123.1, 121.6, 121.0, 114.4, 110.5, 64.6, 56.8, 50.8, 45.0, 42.6, 40.9, 35.9, 28.7; HRMS (ESI+) m/z for C₁₆H₁₉ON₂ [M+H]+: caled 255.14919, found 255.15004.

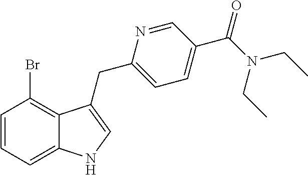 [SMILES: CCN(CC)C(=O)c1ccc(Cc2c[nH]c3cccc(Br)c23)nc1]

6-((4-Bromo-1H-indol-3-yl)methyl)-N,N-diethylnicotinamide. A solution of methyl 6-((4-bromo-1H-indol-3-yl) methyl)nicotinate (0.300 g, 0.869 mmol) and KOH (0.054 g, 0.96 mmol) in EtOH/H₂O (3:1, 5 mL) was heated to 70° C. Upon completion, the solution was concentrated, and HBTU (0.673 g, 1.74 mmol) and DMF (8 mL) were added. The solution was stirred at rt for 1 h followed by addition of diethylamine (0.27 mL, 2.6 mmol). The solution was stirred at rt for 2 h. The solution was diluted with EtOAc (40 mL) and washed with 1:1 H₂O/brine (5×16 mL). The organic phase was dried (MgSO₄), filtered, and concentrated. Purification by chromatography on SiO₂ (EtOAc with 1% Et₃N) afforded the product as a beige semi-solid (0.270 g, 0.699 mmol, 80%): IR (ATR) νₘₐₓ 3205, 2973, 2933, 1609, 1596, 1560, 1489, 1470, 1425, 1376, 1336, 1314, 1289, 1251, 1216, 1196, 1184, 1141, 1100, 1069, 1042, 1027, 944, 913, 877, 803, 767, 734 cm⁻¹; ¹H NMR (500 MHz, CDCl₃) δ 9.89 (d, J=12.5 Hz, 1H), 8.59 (d, J=1.5 Hz, 1H), 7.53 (dd, J=8.0, 2.0 Hz, 1H), 7.18 (d, J=8.0 Hz, 1H), 7.14 (d, J=7.5, 1H), 7.06 (d, J=8.5 Hz, 1H), 6.88 (t, J=7.0 Hz, 1H), 6.80 (d, J=2.0 Hz, 1H), 4.55 (s, 2H), 3.53 (m, 2H), 3.26 (m, 2H), 1.23 (m, 3H), 1.10 (m, 3H); ¹³C NMR (125 MHz, CDCl₃) δ 169.2, 163.5, 146.3, 138.0, 134.8, 130.0, 125.7, 125.3, 123.5, 122.7, 122.6, 113.9, 112.6, 110.9, 43.6, 39.7, 34.8, 14.3, 12.9; HRMS (ESI+) m/z calcd for C₁₉H₂₁ON₃Br [M+H]⁺ 386.08625, found 386.08774.

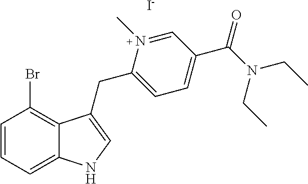

2-((4-Bromo-1H-indol-3-yl) methyl)-5-(diethylcarbamoyl)-1-methylpyridin-1-ium iodide. A solution of 6-((4-bromo-1H-indol-3-yl) methyl)-N,N-diethylnicotinamide (1.88 g, 4.66 mmol) and iodomethane (1.45 mL, 23.3 mmol) in MeCN (47 mL) was stirred at 50° C. for 24 h in a closed tube. Upon completion, the solution was concentrated and carried forward without further purification. An analytical sample was purified by chromatography on C18 (10-50% MeCN/H₂O over 20 CV) to afford the product as a yellow solid: mp 94-96° C.; IR (ATR) νₘₐₓ 3194, 2971, 1624, 1561, 1523, 1476, 1424, 1384, 1334, 1291, 1270, 1180, 1164, 1141, 1102, 1072, 1043, 950, 914, 827, 804, 780, 764, 738, 676 cm⁻¹; ¹H NMR (500 MHz, DMSO-d₆) δ 9.23 (d, J=2.0 Hz, 1H), 8.41 (dd, J=8.5, 1.5 Hz, 1H), 7.49 (d, J=2.5 Hz, 1H), 7.49 (s, 1H), 7.19 (dd, J=7.5, 2.0 Hz, 1H), 7.06 (t, J=8.0 Hz, 1H), 4.73 (s, 2H), 4.41 (s, 3H), 3.47 (q, J=7.0 Hz, 2H), 3.23 (q, J=7.0 Hz, 2H), 1.17 (t, J=7.0 Hz, 3H), 1.06 (t, J=7.0 Hz, 3H); ¹³C NMR (125 MHz, DMSO-d₆) δ 163.8, 159.4, 144.5, 142.1, 138.0, 133.6, 127.9, 127.7, 124.2, 123.1, 123.0, 112.5, 111.8, 106.3, 45.9, 43.2, 29.8, 13.9, 12.6; HRMS (ESI+) m/z calcd for C₂₀H₂₃ON₃Br [M+H]⁺ 400.10190, found 400.10237.

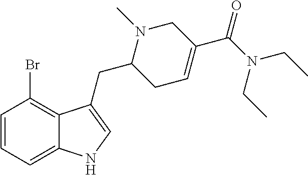 [SMILES: CCN(CC)C(=O)C1=CCC(Cc2c[nH]c3cccc(Br)c23)N(C)C1]

6-((4-Bromo-1H-indol-3-yl)methyl)-N,N-diethyl-1-methyl-1,2,5,6-tetrahydropyridine-3-carboxamide. To a solution of 2-((4-bromo-1H-indol-3-yl) methyl)-5-(diethylcarbamoyl)-1-methylpyridin-1-ium iodide (1.90 g, 3.60 mmol) in MeOH (36 mL) was added NaBH₄ (0.408 g, 10.8 mmol) in 6 equal portions (68 mg) over the course of 1 h at 0° C. The solution was stirred for an additional 30 min. Upon completion, the solution was diluted with H₂O (50 mL) and CH₂Cl₂ (100 mL). The aqueous layer was extracted with CH₂Cl₂ (2×25 mL), and the combined organic layers were washed with sat. aq. NaCl (30 mL), dried (MgSO₄), filtered, and concentrated. Purification by chromatography on SiO₂ (19:1-9:1 EtOAc: MeOH w/1% Et₃N) afforded the product as a tan oil that became solid under vacuum (1.26 g, 3.13 mmol, 87%): mp 76-78° C.; IR (ATR) νₘₐₓ 3201, 2973, 2929, 2790, 1736, 1497, 1477, 1456, 1427, 1380, 1363, 1335, 1311, 1216, 1185, 1140, 1125, 1068, 1043, 1022, 941, 912, 766, 737, 697, 669 cm⁻¹; ¹H NMR (500 MHz, CDCl₃) δ 8.81 (d, J=19.0 Hz, 1H), 7.29 (d, J=8.0 Hz, 1H), 7.25 (d, J=7.5 Hz, 1H), 7.02 (s, 1H), 6.97 (t, J=7.5 Hz, 1H), 5.79 (d, J=2.0 Hz, 1H), 3.54 (dd, J=10.0, 4.0 Hz, 1H), 3.46 (dd, J=15.0, 2.0 Hz, 1H), 3.41 (q, J=7.0 Hz, 4H), 3.33 (dd, J=15.0, 2.0 Hz, 1H), 3.20-3.16 (m, 1H), 2.84 (dd, J=14.0, 10.0 Hz, 1H), 2.57 (s, 3H), 2.14-2.03 (m, 2H), 1.14 (t, J=7.0 Hz, 6H); ¹³C NMR (125 MHz, CDCl₃) δ ¹³C NMR (125 MHz, CDCl₃) δ 170.8, 137.9, 132.9, 125.6, 125.0, 124.0, 123.9, 122.7, 114.3, 113.6, 110.9, 58.2, 53.4, 43.0, 40.5, 39.2, 27.2, 26.7, 14.5, 13.0; HRMS (ESI+) m/z calcd for C₂₀H₂₇ON₃Br [M+H]⁺ 404.13320, found 404.13512.

In view of the many possible embodiments to which the principles of the disclosed invention may be applied, it should be recognized that the illustrated embodiments are only preferred examples of the invention and should not be taken as limiting the scope of the invention.

1. A compound, or a pharmaceutically acceptable salt thereof, of formula I: 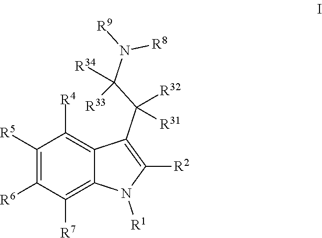
wherein R¹, R⁸, R⁹, R³¹, R³², R³³, and R³⁴ are each independently H, C₁-C₆ alkyl, or substituted C₁-C₆ alkyl; and
R², R⁴, R⁵, R⁶, and R⁷ are each independently H, F, Cl, Br, —OR¹, —NR⁸R⁹, C₁-C₆ alkyl, or substituted C₁-C₆ alkyl.

2. The compound of claim 1, wherein R⁸ and R⁹ are each independently C₁-C₆ alkyl.

3. The compound of claim 1, wherein at least one of R⁴, R⁵, R⁶, and R⁷ is F, Cl, or Br, and the remaining R⁴, R⁵, R⁶, and R⁷ are each H.

4. The compound of claim 1, wherein R¹ is N-heteroaryl-substituted C₁-C₆ alkyl.

5. A compound, or a pharmaceutically acceptable salt thereof, of formula II: 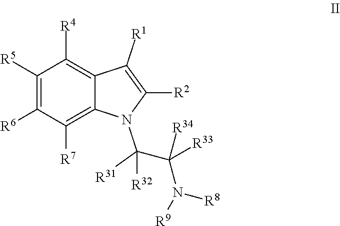
wherein R¹, R⁸, R⁹, R³¹, R³², R³³, and R³⁴ are each independently H, C₁-C₆ alkyl, or substituted C₁-C₆ alkyl; and
R², R⁴, R⁵, R⁶, and R⁷ are each independently H, F, Cl, Br, —OR¹, —NR⁸R⁹, C₁-C₆ alkyl, or substituted C₁-C₆ alkyl.

6. The compound of claim 5, wherein R⁸ and R⁹ are each independently C₁-C₆ alkyl.

7. The compound of claim 5, wherein at least one of R⁴, R⁵, R⁶, and R⁷ is F, Cl, or Br, and the remaining R⁴, R⁵, R⁶, and R⁷ are each H.

8. The compound of claim 5, wherein R¹ is N-heteroaryl-substituted C₁-C₆ alkyl.

9. A compound, or a pharmaceutically acceptable salts thereof, of formula III: 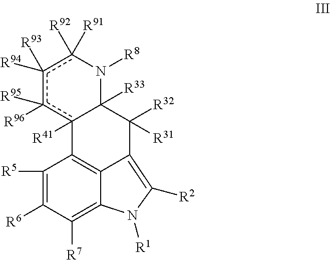
wherein R¹ and R⁸ are each independently H, C₁-C₆ alkyl, or substituted C₁-C₆ alkyl;
R², R⁵, R⁶, R⁷, R⁴¹, R⁹³, R⁹⁴, R⁹⁵, and R⁹⁶ are each independently H, F, Cl, Br, —OR¹, —NR⁸R⁹, —CR¹R¹OR¹, —CO₂R¹, —CONR⁸R⁹, C₁-C₆ alkyl, or substituted C₁-C₆ alkyl;
R⁹, R³¹, R³², R³³, R⁹¹, and R⁹² are each independently H, —CR¹R¹OR¹, —CO₂R¹, —CONR⁸R⁹, C₁-C₆ alkyl, or substituted C₁-C₆ alkyl; and
where each bond represented by [symbol] is a single or double bond as needed to satisfy valence requirements, and where each of R⁴¹, R⁹⁵, R⁹⁴, and R⁹¹ may or may not be present as needed to satisfy valency requirements.

10. The compound of claim 9, wherein the compound is of formula IIIa

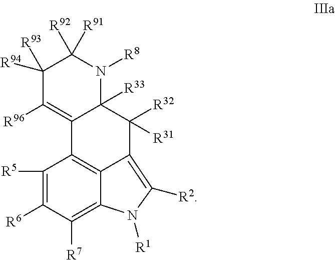

11. The compound of claim 9, wherein the compound is of formula IIIb

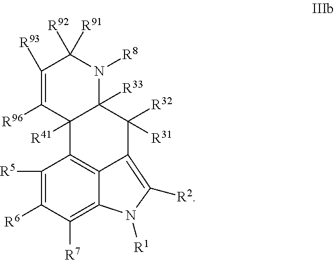

12. The compound of claim 9, wherein the compound is of formula IIIc

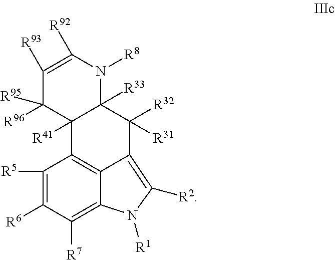

13. The compound of claim 9. wherein the compound is of formula IIId

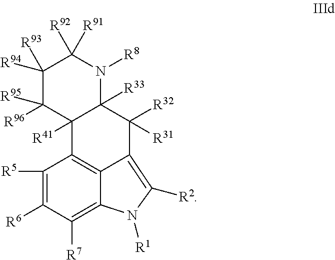

14. The compound of claim 9, wherein R⁸ is methyl.

15. The compound of claim 9, wherein R⁹³ is —CR¹R¹OR¹, wherein each R¹ is H.

16. The compound of claim 9, wherein each of R¹, R², R⁵, R⁶, R⁷, R³¹, R³² and R³³ is H.

17. A compound, or a pharmaceutically acceptable salt thereof, of formula IV: 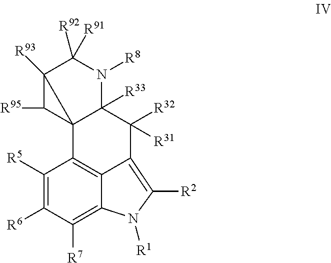
wherein R¹ and R⁸ are each independently H, C₁-C₆ alkyl, or substituted C₁-C₆ alkyl;
R², R⁵, R⁶, R⁷, R⁹³, and R⁹⁵ are each independently H, F, Cl, Br, —OR¹, —NR⁸R⁹, —CR¹R¹OR¹, —CO₂R¹, —CONR⁸R⁹, C₁-C₆ alkyl, or substituted C₁-C₆ alkyl; and
R⁹, R³¹, R³², R³³, R⁹¹, and R⁹² are each independently H, —CR¹R¹OR¹, —CO₂R¹, —CONR⁸R⁹, C₁-C₆ alkyl, or substituted C₁-C₆ alkyl.

18. The compound of claim 17, wherein R⁸ is methyl.

19. A compound, or a pharmaceutically acceptable salt thereof, of formula V: 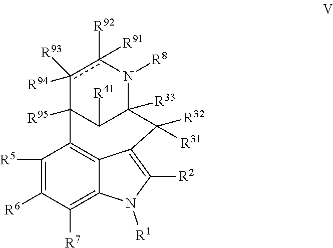
wherein R¹ and R⁸ are each independently H, C₁-C₆ alkyl, or substituted C₁-C₆ alkyl;
R², R⁵, R⁶, R⁷, R⁴¹, R⁹³, R⁹⁴ and R⁹⁵ are each independently H, F, Cl, Br, —OR¹, —NR⁸R⁹, —CR¹R¹OR¹, —CO₂R¹, —CONR⁸R⁹, C₁-C₆ alkyl, or substituted C₁-C₆ alkyl;
R⁹, R³¹, R³², R³³, R⁹¹, and R⁹² are each independently H, —CR¹R¹OR¹, —CO₂R¹, —CONR⁸R⁹, C₁-C₆ alkyl, or substituted C₁-C₆ alkyl; and
where each bond represented by [symbol] is a single or double bond as needed to satisfy valence requirements, and where each of R⁹⁴ and R⁹¹ may or may not be present as needed to satisfy valency requirements.

20. The compound of claim 19, wherein R⁸ is methyl.

21. The compound of claim 19, wherein R⁹³ is —CONR⁸R⁹, wherein R⁸ and R⁹ are each C₁-C₆ alkyl, and R⁹⁴ is H.

22. A method for improving the well-being of a subject, comprising administering a therapeutically effective amount of a compound of claim 1 to the subject in need thereof.

23. A method for treating a neurological condition in a subject, comprising administering a therapeutically effective amount of a compound of claim 1 to the subject in need thereof.
